(19) United States
(12) Patent Application Publication
(10) Pub. No.: US 2026/0356982 A1
(43) Pub. Date: Nov. 12, 2026

(54) ANTI-FLAG ANTIBODIES

(71) Applicant: Currus Biologics Pty Ltd, Melbourne (AU)

(72) Inventor: Samantha Cobb, Melbourne (AU)

(73) Assignee: Currus Biologics Pty Ltd, Melbourne (AU)

(21) Appl. No.: 19/112,558

(22) PCT Filed: Sep. 20, 2023
(86) PCT No.: PCT/AU2023/050903

§ 371 Date: Mar. 17, 2025

(30) Foreign Application Priority Data
Sep. 20, 2022 (AU) 2022902712

(51) Int. Cl. C07K 16/44; A61K 47/68

(52) U.S. Cl. CPC C07K 16/44; A61K 47/6835; C07K 2317/24; C07K 2317/41; C07K 2317/565; C07K 2317/567; C07K 2317/622; C07K 2317/92

(57) ABSTRACT

The invention relates to antigen binding proteins and related fragments thereof for binding to FLAG tag, to production of said antigen binding proteins and fragments and to use of said antibodies and fragments.

### FIELD OF THE INVENTION

The invention relates to antigen binding proteins and related fragments thereof for binding to FLAG tag, including humanised antigen binding proteins and related fragments thereof, to production of said antigen binding proteins and fragments thereof, and to uses of said antibodies and fragments thereof.

### RELATED APPLICATION

This application claims priority from Australian provisional application AU 2022902712, the entire contents of which are hereby incorporated by reference.

### BACKGROUND OF THE INVENTION

The “FLAG” tag, (also known as the FLAG epitope) is an artificial antigen peptide tag that is frequently incorporated into proteins in order to facilitate detection and/or affinity purification of a target protein. It is one of the most commonly used protein tags in laboratories world-wide.

Antibodies for binding to FLAG tag are known however such antibodies have been generated in mice or other rodents consequently are not suitable for clinical use. While the use of mouse anti-FLAG antibodies is often acceptable for the detection or purification of recombinant proteins in in vitro systems, the use of mouse antibodies is not always appropriate for the detection of or binding to FLAG tag in in vivo systems.

Consequently there is a need for new and/or improved anti-FLAG antibodies.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to antigen binding proteins comprising an antigen binding domain for binding to FLAG tag. In particular, the invention relates to humanised anti-FLAG antibodies, and FLAG-binding fragments thereof.

The invention provides an antigen binding protein for binding to FLAG tag, the antigen binding protein comprising an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1 below.

In any embodiment, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

In any embodiment, an antigen binding protein of the invention is capable of specifically binding to a FLAG tag or variant thereof (such as defined in SEQ ID NOs: 11 and 29 or as otherwise defined herein). The antigen binding proteins of the invention are preferably capable of specifically binding to protein domains comprising multiples of the FLAG tag sequences (eg 2× FLAG, 3× FLAG etc).

In any embodiment, the antigen binding proteins of the invention are for specifically binding to a peptide tag that contains or consists of the sequence DYK, preferably the sequence DYKD (SEQ ID NO: 11). In addition to these sequences, other amino acids can be present, preferably hydrophilic amino acids for example R (Arg), D (Asp), E (Glu) and K (Lys) and/or amino acids with aromatic side chains for example Y (Tyr), F (Phe), H (His) and W (Trp).

In a preferred embodiment, the antigen binding proteins are capable of specifically binding to a FLAG-tag that contains or consists of the sequence GDYKDDDDKG (SEQ ID NO: 29), DYKDDDDK (SEQ ID NO: 30), MDYKDDDDK (SEQ ID NO: 31), DFKDDDK (SEQ ID NO: 32), DYKAFDNL (SEQ ID NO: 33), DYKDHDG (SEQ ID NO: 34), MDFKDDDDK (SEQ ID NO: 35), MDYKAFDNL (SEQ ID NO: 36), DYKDHDI (SEQ ID NO: 37), DYKDH (SEQ ID NO: 38), DYKDD (SEQ ID NO: 39), DYKDHD (SEQ ID NO: 40) and/or DYKDDD (SEQ ID NO: 41). The most preferred sequence is DYKDDDDK (SEQ ID NO: 30).

As used herein, the term FLAG-tag also refers to modified FLAG-tags, which are derived from the FLAG-tags described above, especially the tag with the sequence DYKDDDDK, by amino acid insertion, deletion or substitution.

Reference herein to a protein or antibody that “binds to” FLAG tag provides literal support for a protein or antibody that “binds specifically to” or “specifically binds to” a FLAG tag.

The antigen binding proteins of the invention are for specifically binding to FLAG tags, particularly in the context of recombinant or therapeutic proteins (including antibodies or fragments thereof or proteins comprising antigen binding domains for use in a therapeutic), or cell-based therapies (cellular immunotherapies including genetically modified cells) comprising a FLAG tag. In certain examples, the antigen binding proteins are useful for binding to a FLAG tag included in chimeric antigen receptors, and related recombinant receptors for expression by a cellular immunotherapeutic (eg: included in CARs for expression by cytotoxic T cells).

In any embodiment, the FLAG tag is present at the N-terminus, the C-terminus or within the protein to which is it intended to bind.

In any embodiment, the invention provides an antigen binding protein for binding to a FLAG tag, wherein the antigen binding protein competitively inhibits the binding to a FLAG tag of an antibody:
comprising a VH comprising a sequence as set forth in SEQ ID NO: 1 and a VL comprising a sequence as set forth in SEQ ID NO: 6;
comprising a VH comprising a sequence as set forth in SEQ ID NO: 2 and a VL comprising a sequence as set forth in SEQ ID NO: 7;
comprising a VH comprising a sequence as set forth in SEQ ID NO: 2 and a VL comprising a sequence as set forth in SEQ ID NO: 8;
comprising a VH comprising a sequence as set forth in SEQ ID NO: 2 and a VL comprising a sequence as set forth in SEQ ID NO: 9;
comprising a VH comprising a sequence as set forth in SEQ ID NO: 2 and a VL comprising a sequence as set forth in SEQ ID NO: 10;
comprising a VH comprising a sequence as set forth in SEQ ID NO: 3 and a VL comprising a sequence as set forth in SEQ ID NO: 7;
comprising a VH comprising a sequence as set forth in SEQ ID NO: 3 and a VL comprising a sequence as set forth in SEQ ID NO: 8;
comprising a VH comprising a sequence as set forth in SEQ ID NO: 3 and a VL comprising a sequence as set forth in SEQ ID NO: 9;
comprising a VH comprising a sequence as set forth in SEQ ID NO: 3 and a VL comprising a sequence as set forth in SEQ ID NO: 10;
comprising a VH comprising a sequence as set forth in SEQ ID NO: 4 and a VL comprising a sequence as set forth in SEQ ID NO: 7;
comprising a VH comprising a sequence as set forth in SEQ ID NO: 4 and a VL comprising a sequence as set forth in SEQ ID NO: 8;
comprising a VH comprising a sequence as set forth in SEQ ID NO: 4 and a VL comprising a sequence as set forth in SEQ ID NO: 9;
comprising a VH comprising a sequence as set forth in SEQ ID NO: 4 and a VL comprising a sequence as set forth in SEQ ID NO: 10;
comprising a VH comprising a sequence as set forth in SEQ ID NO: 5 and a VL comprising a sequence as set forth in SEQ ID NO: 7;
comprising a VH comprising a sequence as set forth in SEQ ID NO: 5 and a VL comprising a sequence as set forth in SEQ ID NO: 8;
comprising a VH comprising a sequence as set forth in SEQ ID NO: 5 and a VL comprising a sequence as set forth in SEQ ID NO: 9; or
comprising a VH comprising a sequence as set forth in SEQ ID NO: 5 and a VL comprising a sequence as set forth in SEQ ID NO: 10.

In any embodiment, the invention provides an antigen binding protein with a CDRH1, a CDRH2 and/or a CDRH3 of an antigen binding domain having a variable heavy chain as defined in any one of SEQ ID NOs: 2 to 5.

In any embodiment, the invention provides an antigen binding protein with a CDRL1, a CDRL2 and/or a CDRL3 of an antigen binding domain having a variable light chain as defined in any one of SEQ ID NOs: 7 to 10.

In any embodiment, the invention provides an antigen binding protein comprising a CDR1, a CDR2 and/or a CDR3 of an antigen binding domain having a variable heavy chain as defined in any one of SEQ ID NOs: 2 to 5, and a CDR1, a CDR2 and/or a CDR3 of an antigen binding domain having a variable light chain as defined in any one of SEQ ID NOs: 7 to 10.

In an embodiment, the antigen binding domain comprises:
(a) and (b):
(a) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 12; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 13; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 14; and
(b) a VL comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 21; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 22; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (c) and (d):
(c) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 42 or 43; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 44; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(d) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 51; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (e) and (f):
(e) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 42 or 43; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 44; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(f) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 64; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (g) and (h):
(g) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 42 or 87; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 88; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(h) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 51; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (i) and (j):
(i) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 42 or 87; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 88; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(j) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 64; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (k) and (l):
(k) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 57; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 58; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(l) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 51; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (m) and (n):
(m) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 57; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 58; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(n) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 64; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (o) and (p):
(o) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 42; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 63; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(p) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 64; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23,
or (q) and (r):
(q) a VH comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 57; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 58; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45; and
(r) a VL comprising a complementarity determining region CDR1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 64; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 52; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 23.

In an embodiment, the antigen binding domain comprises:
(a) and (b):
(a) a VH comprising a complementarity determining region (CDR) 1 comprising or consisting of an amino acid sequence of SEQ ID NO: 12; a CDR2 comprising or consisting of an amino acid sequence of SEQ ID NO: 13; a CDR3 comprising or consisting of an amino acid sequence of SEQ ID NO: 14; and
(b) a VL comprising a complementarity determining region (CDR) 1 comprising or consisting of an amino acid sequence SEQ ID NO: 21; a CDR2 comprising or consisting of an amino acid sequence of SEQ ID NO: 22; a CDR3 comprising or consisting of an amino acid sequence SEQ ID NO: 23,
or (c) and (d):
(c) a VH comprising a complementarity determining region CDR1 comprising or consisting of an amino acid sequence of SEQ ID NOs: 42 or 43; a CDR2 comprising or consisting of an amino acid sequence of SEQ ID NO: 44; a CDR3 comprising or consisting of an amino acid sequence of SEQ ID NO: 45; and
(d) a VL comprising a complementarity determining region CDR1 comprising or consisting of an amino acid sequence of SEQ ID NO: 51; a CDR2 comprising or consisting of an amino acid sequence of SEQ ID NO: 52; a CDR3 comprising or consisting of an amino acid sequence of SEQ ID NO: 23,
or (e) and (f):
(e) a VH comprising a complementarity determining region CDR1 comprising or consisting of an amino acid sequence of SEQ ID NOs: 42 or 43; a CDR2 comprising or consisting of an amino acid sequence of SEQ ID NO: 44; a CDR3 comprising or consisting of an amino acid sequence of SEQ ID NO: 45; and
(f) a VL comprising a complementarity determining region CDR1 comprising or consisting of an amino acid sequence of SEQ ID NO: 64; a CDR2 comprising or consisting of an amino acid sequence SEQ ID NO: 52; a CDR3 comprising or consisting of an amino acid sequence of SEQ ID NO: 23,
or (g) and (h):
(g) a VH comprising a complementarity determining region CDR1 comprising or consisting of an amino acid sequence of SEQ ID NOs: 42 or 87; a CDR2 comprising or consisting of an amino acid sequence of SEQ ID NO: 88; a CDR3 comprising or consisting of an amino acid sequence of SEQ ID NO: 45; and
(h) a VL comprising a complementarity determining region CDR1 comprising or consisting of an amino acid sequence of SEQ ID NO: 51; a CDR2 comprising or consisting of an amino acid sequence of SEQ ID NO: 52; a CDR3 comprising or consisting of an amino acid sequence of SEQ ID NO: 23,
or (i) and (j):
(i) a VH comprising a complementarity determining region CDR1 comprising or consisting of an amino acid sequence of SEQ ID NOs: 42 or 87; a CDR2 comprising or consisting of an amino acid sequence of SEQ ID NO: 88; a CDR3 comprising or consisting of an amino acid sequence of SEQ ID NO: 45; and
(j) a VL comprising a complementarity determining region CDR1 comprising or consisting of an amino acid sequence of SEQ ID NO: 64; a CDR2 comprising or consisting of an amino acid sequence of SEQ ID NO: 52; a CDR3 comprising or consisting of an amino acid sequence of SEQ ID NO: 23,
or (k) and (l):
(k) a VH comprising a complementarity determining region CDR1 comprising or consisting of an amino acid sequence of SEQ ID NO: 57; a CDR2 comprising or consisting of an amino acid sequence of SEQ ID NO: 58; a CDR3 comprising or consisting of an amino acid sequence of SEQ ID NO: 45; and
(l) a VL comprising a complementarity determining region CDR1 comprising or consisting of an amino acid sequence of SEQ ID NO: 51; a CDR2 comprising or consisting of an amino acid sequence of SEQ ID NO: 52; a CDR3 comprising or consisting of an amino acid sequence of SEQ ID NO: 23,
or (m) and (n):
(m) a VH comprising a complementarity determining region CDR1 comprising or consisting of an amino acid sequence of SEQ ID NO: 57; a CDR2 comprising or consisting of an amino acid sequence of SEQ ID NO: 58; a CDR3 comprising or consisting of an amino acid sequence of SEQ ID NO: 45; and
(n) a VL comprising a complementarity determining region CDR1 comprising or consisting of an amino acid sequence of SEQ ID NO: 64; a CDR2 comprising or consisting of an amino acid sequence of SEQ ID NO: 52; a CDR3 comprising or consisting of an amino acid sequence of SEQ ID NO: 23,
or (o) and (p):
(o) a VH comprising a complementarity determining region CDR1 comprising or consisting of an amino acid sequence of SEQ ID NO: 42; a CDR2 comprising or consisting of an amino acid sequence of SEQ ID NO: 63; a CDR3 comprising or consisting of an amino acid sequence of SEQ ID NO: 45; and
(p) a VL comprising a complementarity determining region CDR1 comprising or consisting of an amino acid sequence of SEQ ID NO: 64; a CDR2 comprising or consisting of an amino acid sequence of SEQ ID NO: 52; a CDR3 comprising or consisting of an amino acid sequence of SEQ ID NO: 23,
or (q) and (r):
(q) a VH comprising a complementarity determining region CDR1 comprising or consisting of an amino acid sequence of SEQ ID NO: 57; a CDR2 comprising or consisting of an amino acid sequence of SEQ ID NO: 58; a CDR3 comprising or consisting of an amino acid sequence of SEQ ID NO: 45; and
(r) a VL comprising a complementarity determining region CDR1 comprising or consisting of an amino acid sequence of SEQ ID NO: 64; a CDR2 comprising or consisting of an amino acid sequence of SEQ ID NO: 52; a CDR3 comprising or consisting of an amino acid sequence of SEQ ID NO: 23.

In any embodiment, FR1, FR2, FR3 and FR4 may refer to framework regions from the variable heavy chain of an antibody (a VH), FR1a, FR2a, FR3a and FR4a may refer to framework regions from the variable light chain of an antibody (a VL), or where FR1, FR2, FR3 and FR4 are framework regions from the VL, FR1a, FR2a, FR3a and FR4a are framework regions from VH. In such examples, the FRs may be referred to as FR H1, FR H2, FR H3, FR H4, FR L1, FR L2, FR L3 and FR L4 as the case may be.

In any embodiment, the invention provides an antigen binding protein with a FR H1, a FR H2, a FR H3 and/or a FR H4 from a human germline, wherein the human germline is IGHV1-46*01 or IGHV7-4-1*02.

In any embodiment, the invention provides an antigen binding protein with a FR L1, a FR L2, a FR L3 and/or a FR L4 from a human germline, wherein the human germline is IGKV2-30*01 or IGKV4-1*01.

In any embodiment, the invention provides an antigen binding protein with a FR H1, a FR H2, a FR H3 and/or a FR H4 from a human germline, wherein the human germline is IGHV1-46*01 or IGHV7-4-1*02, and a FR L1, a FR L2, a FR L3 and/or a FR L4 from a human germline, wherein the human germline is IGKV2-30*01 or IGKV4-1*01.

In any embodiment, the antigen binding protein comprises a VH that has a greater than 80%, 81%, 82%, 8%, 84%, 85%, 86%, 87%, 88%, 89%, 90%, 91%, 92% identity to human, preferably the % identity to human is calculated as described in Example 1, and/or a VL that has a greater than 80%, 81%, 82%, 8%, 84%, 85%, 86%, 87%, 88%, 89%, 90%, 91%, 92% identity to human, preferably the % identity to human is calculated as described in Example 1.

In any embodiment, the invention provides an antigen binding protein with a FR H1, a FR H2, a FR H3 and/or a FR H4 of an antigen binding domain having a variable heavy chain as defined in any one of SEQ ID NOs: 2 to 5.

In any embodiment, the invention provides an antigen binding protein with a FR L1, a FR L2, a FR L3 and/or a FR L4 of an antigen binding domain having a variable light chain as defined in any one of SEQ ID NOs: 7 to 10.

In any embodiment, the invention provides an antigen binding protein comprising a FR1, a FR2, a FR3 and/or a FR4 of an antigen binding domain having a variable heavy chain as defined in any one of SEQ ID NOs: 2 to 5, and a FR1, a FR2, a FR3 and/or a FR4 of an antigen binding domain having a variable light chain as defined in any one of SEQ ID NOs: 7 to 10.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any (a) above and a VL comprising a CDR1, CDR2 and CDR3 as defined in any (b) above,
and any one of B., C., D., E., F., G., H., I., J., K., L., M., N., O., P., Q., or R.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 65, 66, 67 and 68 respectively, and for VL FRs the reference sequences are set forth in SEQ ID NOs: 69, 70, 71 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 69, 70, 71 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NO: 84, 85, 86 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 24, 26, 27 and 28 respectively,
or
F. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 24, 25, 27 and 28 respectively,
or
G. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 69, 70, 71 and 28 respectively,
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 84, 85, 86 and 28 respectively,
or
I. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 24, 26, 27 and 28 respectively,
or
J. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 24, 25, 27 and 28 respectively,
or
K. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 69, 70, 71 and 28 respectively,
or
L. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 84, 85, 86 and 28 respectively,
or
M. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 24, 26, 27 and 28 respectively,
or
N. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 24, 25, 27 and 28 respectively,
or
O. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 81, 83 and 20 respectively, and for VL FRs the reference sequences are set forth in SEQ ID NOs: 69, 70, 71 and 28 respectively,
or
P. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 81, 83 and 20 respectively, and for VL FRs the reference sequences are set forth in SEQ ID NOs: 84, 85, 86 and 28 respectively,
or
Q. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 80, 81, 83 and 20 respectively, and for VL FRs the reference sequences are set forth in SEQ ID NO: 24, 26, 27 and 28 respectively,
or
R. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 80, 81, 83 and 20 respectively, and for VL FRs the reference sequences are set forth in SEQ ID NOs: 24, 25, 27 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (c) above and a VL comprising a CDR1, CDR2 and CDR3 as defined in any of (d) above,
and any one of B., C., D., or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 53, 55, 56 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 53, 54, 56 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 53, 55, 56 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 53, 54, 56 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (e) above and a VL comprising a CDR1, CDR2 and CDR3 as defined in any of (f) above,
and any one of B., C., D. or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 75, 76, 77 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 92, 93, 94 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 75, 76, 77 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 92, 93, 94 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (g) above and a VL comprising a CDR1, CDR2 and CDR3 as defined in any of (h) above,
and any one of B., C., D. or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 53, 55, 56 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 53, 54, 56 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 53, 55, 56 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 53, 54, 56 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (i) above and a VL comprising a CDR1, CDR2 and CDR3 as defined in any of (j) above,
and any one of B., C., D. or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 75, 76, 77 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 92, 93, 94 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 75, 76, 77 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 92, 93, 94 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (k) above and a VL comprising a CDR1, CDR2 and CDR3 as defined in any of (l) above,
and any one of B., C., D., E., F., G., H. or I.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 53, 55, 56 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 53, 54, 56 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 53, 55, 56 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 53, 54, 56 and 28 respectively,
or
F. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 53, 55, 56 and 28 respectively,
or
G. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 53, 54, 56 and 28 respectively,
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 53, 55, 56 and 28 respectively,
or
I. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 53, 54, 56 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (m) above and a VL comprising a CDR1, CDR2 and CDR3 as defined in any of (n) above,
and any one of B., C., D., E., F., G., H., or I.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 75, 76, 77 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 99, 100, 101 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 75, 76, 77 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 99, 100, 101 and 28 respectively,
or
F. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 75, 76, 77 and 28 respectively,
or
G. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 99, 100, 101 and 28 respectively,
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 75, 76, 77 and 28 respectively,
or
I. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 99, 100, 101 and 28 respectively.

In an embodiment, the present invention provides an antigen binding protein comprising an antigen binding domain, wherein the antigen binding domain comprises:
A. a VH comprising a complementarity determining region (CDR) 1, CDR2 and CDR3 as defined in any of (o) above and a VL comprising a CDR1, CDR2 and CDR3 as defined in any of (p) above, and a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 72, 73, 74 and 68 respectively, and for VL FRs the reference sequences are set forth in SEQ ID NOs: 75, 76, 77 and 28 respectively,
or
B. a VH comprising a complementarity determining region (CDR) 1, CDR2 and CDR3 as defined in any of (q) above and a VL comprising a CDR1, CDR2 and CDR3 as defined in any of (r) above and a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 78, 79 and 68 respectively, and for VL FRs the reference sequences are set forth in SEQ ID NOs: 75, 76, 77 and 28 respectively.

In any embodiment, the antigen binding domain comprises a variable heavy chain comprising the amino acid sequence as set forth in any one of SEQ ID NOs: 2 to 5, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto.

In any embodiment, the antigen binding domain comprises a variable light chain comprising the amino acid sequence as set forth in any one of SEQ ID NOs: 7 to 10; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto.

In any embodiment, the antigen binding domain comprises a variable heavy chain comprising the amino acid sequence as set forth in any one of SEQ ID NOs: 2 to 5 and a variable light chain comprising the amino acid sequence as set forth in any one of SEQ ID NOs: 7 to 10; or sequences at least about 80%, at least about 85%, at least about 90%, at least about 91%, at least about 92%, at least about 93%, at least about 94%, at least about 95%, at least about 96%, at least about 97%, at least about 98%, or at least about 99% identical thereto.

As described herein, the antigen binding protein may be in the form of:
(i) a single domain antibody (sdAb);
(ii) a single chain Fv fragment (scFv);
(iii) a dimeric scFv (di-scFv);
(iv) one of (ii) or (iii) linked to a constant region of an antibody, Fc or a heavy chain constant domain (CH) 2 and/or CH3;
(v) one of (i) to (iv) linked to a protein that binds to an immune effector cell;
(vi) one of (i) to (iv) linked to a modified immune cell receptor, such as a modified T cell receptor; or
(vii) one of (i) to (iv) in the context of a chimeric antigen receptor (CAR) or variant T cell receptor.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3; or
(ix) one of (i) to (vii) linked to a protein that binds to an immune effector cell.
(x) one of (i) to (vii) linked to a protein that binds to an immune effector cell;
(xi) one of (i) to (vii) linked to a modified immune cell receptor, such as a modified T cell receptor; or
(xiii) one of (i) to (vii) in the context of a chimeric antigen receptor (CAR) or variant T cell receptor (including in the context of a universal CAR system for use with polypeptides comprising antigen binding domains for binding antigens on the surface of a target cell).

The foregoing antigen binding proteins can also be referred to as antigen binding domains of antibodies.

Preferably, an antigen binding protein as described herein is an antibody or antigen binding fragment thereof. Typically, the antigen binding protein is an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein

As used herein the antigen binding protein may be a variable domain.

The invention provides an antigen binding protein including, consisting essentially of or consisting of an amino acids sequence of (in order of N to C terminus or C to N terminus):
SEQ ID NO: 2 and 10;
SEQ ID NO: 3 and 9;
SEQ ID NO: 2 and 9;
SEQ ID NO: 2 and 7;
SEQ ID NO: 2 and 8;
SEQ ID NO: 3 and 7;
SEQ ID NO: 3 and 8;
SEQ ID NO: 3 and 10;
SEQ ID NO: 4 and 7;
SEQ ID NO: 4 and 8;
SEQ ID NO: 4 and 9;
SEQ ID NO: 4 and 10;
SEQ ID NO: 5 and 7;
SEQ ID NO: 5 and 8;
SEQ ID NO: 5 and 9; or
SEQ ID NO: 5 and 10.

As used herein, the complementarity determining region sequences (CDRs) of an antigen binding protein of the invention are defined according to the IMGT, Chothia or Kabat numbering system.

The invention provides an antigen binding protein as described herein wherein an amino acid sequence forming one or more of FR1, CDR1, FR2, CDR2, FR3, CDR3 and FR4 is a human sequence.

The invention provides an anti-FLAG tag antigen binding protein, immunoglobulin variable domain, antibody, dab, scFv, Fab, Fab′, F(ab′)2, Fv fragment, diabody, triabody, linear antibody, single-chain antibody molecule, or multispecific antibody comprising an antigen binding protein having a sequence as described herein, or including a CDR and/or FR sequence as described herein.

The invention provides a diabody or triabody including an antigen binding protein having a sequence as described herein, or comprising a CDR and/or FR sequence as described herein.

The invention provides a fusion protein comprising an antigen binding protein, immunoglobulin variable domain, antibody, dab, scFv, Fab, Fab′, F(ab′)2, Fv fragment, diabody, triabody, linear antibody, single-chain antibody molecule, or multispecific antibody as described herein.

In one embodiment, an antigen binding protein of the invention may be included as a component of a chimeric antigen protein (CAR) or variant T cell receptor. In such embodiments it will be appreciated that the CAR or variant T cell receptor that is generated comprising the antigen binding protein of the invention may be referred to as an “indirect CAR” (sometimes also referred to as a universal CAR). In an indirect CAR system, the antigen binding domain of the CAR does not directly bind to a target antigen on a target cell, but instead binds to an intermediate, wherein the intermediate comprise an antigen binding domain for directly binding the target cell. Examples of a CAR that recognise cells by way of an intermediate are known in the art, for example European patent application EP 2651442.

Thus, in the context of an indirect CAR, the antigen binding protein of the invention may be for binding to a FLAG tag present on an intermediate and wherein the intermediate also an antigen binding domain for binding a target cell (eg a cancer cell). The intermediate provides the specificity for the target cell (such as a cancer cell) whilst the genetically modified cell having the CAR provides the efficacy and directs an immune response against the target cell. The intermediate may be a molecule such as a probe that binds or interacts directly with a target antigen on a target cell, and also comprises a FLAG tag. Non-limiting examples of such probes include antibodies, fusion proteins, a Fab of an antibody, a scFv, a soluble engineered TCR or an aptamer. The term “aptamer” refers to any oligonucleic acid, polynucleic acid, peptide or polypeptide which specifically binds to, or preferentially forms a complex with, a target.

The invention also provides a conjugate in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody or fusion protein as described herein conjugated to a label or a cytotoxic agent.

The invention provides an antibody for binding to an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, or conjugate as described herein.

In aspects of the invention directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
wherein the first and second polypeptides associate to form an antigen binding protein of the present invention.

Examples of cells of the present invention include bacterial cells, yeast cells, insect cells or mammalian cells.

The invention provides a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides a vector comprising a nucleic acid described herein. Preferably, the nucleic acid has a nucleotide sequence that encodes any one or more of the amino acid sequences corresponding to SEQ ID NO: 2 to 5 and 7 to 10.

The invention provides a cell comprising a vector or nucleic acid described herein.

In another embodiment there is provided an animal or tissue derived therefrom comprising a cell described herein.

The invention provides a pharmaceutical composition comprising an antigen binding protein, or including a CDR and/or FR sequence as described herein, or an immunoglobulin variable domain, antibody, dab, scFv, Fab, Fab′, F(ab′)2, Fv fragment, diabody, triabody, linear antibody, single-chain antibody molecule, or multispecific antibody, fusion protein, or conjugate as described herein and a pharmaceutically acceptable carrier, diluent or excipient.

The invention provides a diagnostic composition comprising an antigen binding protein, or including a CDR and/or FR sequence as described herein, or antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein, a diluent and optionally a label.

The invention provides a kit or article of manufacture comprising an antigen binding protein, or including a CDR and/or FR sequence as described herein or an immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides use of a sequence according to one or more of CDR1, CDR2, CDR3, FR1, FR2, FR3 and FR4 as described herein to produce an antigen binding protein for binding to a FLAG tag.

The invention provides a library of nucleic acid molecules produced from the mutation of an antigen binding protein or a CDR and/or FR sequence as described herein, wherein at least one nucleic acid molecule in said library encodes an antigen binding protein for binding to a FLAG tag.

The invention provides a method for producing an antigen binding protein for binding to a FLAG tag as described herein comprising expressing a nucleic acid as described herein in a cell or animal as described herein.

In any aspect of the present invention, the antigen binding protein comprises an Fc region that is engineered to have enhanced capacity to induce antibody-dependent cell-mediated cytotoxicity (ADCC). Preferably, the enhanced capacity to induce ADCC is conferred by mutation, deletion or modification of amino acids in the Fc region which interact with an Fc receptor. Preferably, the amino acids that are mutated, deleted or modified are at position 239, 330, and/or 332 as per SEQ ID NO: 60 (where alanine is position 118) or at an equivalent position to 239, 330 and/or 332. Preferably, the amino acids are mutated to S239D, A330L and 1332E. Typically, the Fc comprises, consists essentially of or consists of an amino acid sequence shown in SEQ ID NO: 62.

In any aspect of the present invention, the antigen binding protein comprises an Fc region that is not engineered to have a reduced capacity to induce antibody-dependent cell mediated cytotoxicity (ADCC). Preferably, the amino acids at position 234, 235, and/or 331 as per SEQ ID NO: 60 (where alanine is position 118) or at an equivalent position to 234, 235 and/or 331 are not F, E and/or S respectively. In other words, the amino acid at position 234 is not F, at position 235 is not E and/or at 331 is not S.

In another embodiment, the antigen binding protein comprises an Fc region that is engineered to:
increase the in vitro or in vivo half-life;
have an increased capacity to induce antibody-dependent cell mediated cytotoxicity (ADCC), antibody-dependent cellular phagocytosis (ADCP) or complement-dependent cytotoxicity;
reduce the effector function; or
increase co-engagement of the antigen binding protein.

The invention provides use of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein in the manufacture of a medicament for the treatment of cancer or a condition or disease associated with expression of FLAG tag.

An antigen binding protein, a protein or antibody as described herein preferably comprises a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

The functional characteristics of an antigen binding protein of the invention will be taken to apply mutatis mutandis to an antibody of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

The invention provides a single domain antibody comprising an antigen binding protein for binding to a FLAG tag.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: ELISA titre of IgG humanised FLAG-tag binding monoclonal antibodies.

FIG. 2: Humanised monoclonal antibodies for binding to FLAG-tagged CARs induce IFN gamma secretion from CAR T cells.

FIG. 3: Alignment of VH and VL regions from parental antibody with humanised variants v4 and v7.

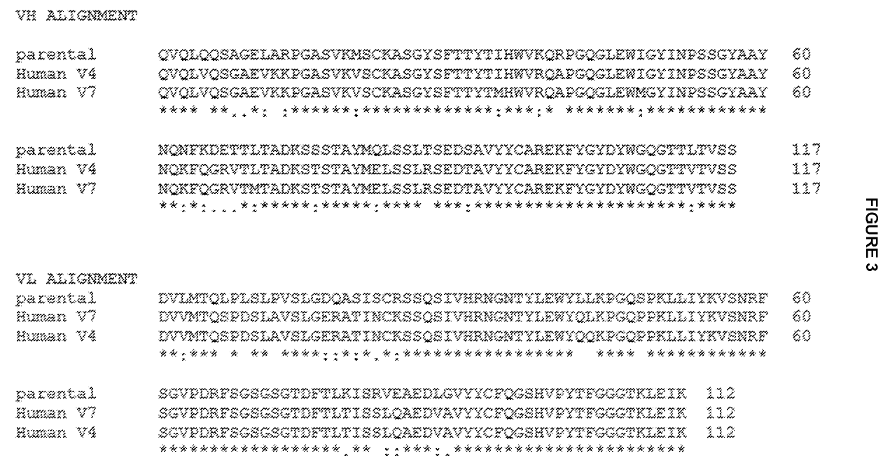

### SEQUENCE INFORMATION

TABLE 1
Sequence information

| Description | SEQ ID NO: | Amino acid sequence |
|---|---|---|
| Parental VH | 1 | QVQLQQSAGELARPGASVKMSCKASGYSFTT |
| (mouse) | | YTIHWVKQRPGQGLEWIGYINPSSGYAAYNQ |
| | | NFKDETTLTADKSSSTAYMQLSSLTSEDSAVY |
| | | YCAREKFYGYDYWGQGTTLTVSS |
| Humanised VH1 | 2 | QVQLVQSGAEVKKPGASVKVSCKASGYSFTT |
| (hu-VH1) | | YTIHWVRQAPGQGLEWIGYINPSSGYAAYNQ |
| Heavy chain variant | | KFQGRVTLTADKSTSTAYMELSSLRSEDTAVY |
| 1 (IGHV1-46*01 | | YCAREKFYGYDYWGQGTTVTVSS |
| germline) | | |
| Humanised VH2 | 3 | QVQLVQSGAEVKKPGASVKVSCKASGYSFTT |
| (hu-VH2) | | YTMHWVRQAPGQGLEWMGYINPSSGYAAYN |
| Heavy chain variant | | QKFQGRVTMTADKSTSTAYMELSSLRSEDTA |
| 2 (IGHV1-46*01 | | VYYCAREKFYGYDYWGQGTTVTVSS |
| germline) | | |
| Humanised VH3 | 4 | QVQLVQSGSELKKPGASVKVSCKASGYSFTT |
| (hu-VH3) | | YTIHWVRQAPGQGLEWIGYINPSSGYAAYNQ |
| Heavy chain variant | | GFTGRFVLSADKSVSTAYLQISSLKAEDTAVYY |
| 3 (IGHV7-4-1*-2 | | CAREKFYGYDYWGQGTTVTVSS |
| germline) | | |
| Humanised VH4 | 5 | QVQLVQSGSELKKPGASVKVSCKASGYSFTT |
| (hu-VH4) | | YTMNWVRQAPGQGLEWMGYINPSSGYAAYN |
| Heavy chain variant | | QGFTGRFVFSADKSVSTAYLQISSLKAEDTAV |
| 4 (IGHV7-4-1*-2 | | YYCAREKFYGYDYWGQGTTVTVSS |
| germline) | | |
| Parental VL | 6 | DVLMTQLPLSLPVSLGDQASISCRSSQSIVHR |
| (mouse) | | NGNTYLEWYLLKPGQSPKLLIYKVSNRFSGVP |
| | | DRFSGSGSGTDFTLKISRVEAEDLGVYYCFQG |
| | | SHVPYTFGGGTKLEIK |
| Humanised VL1 | 7 | DVVMTQSPLSLPVTLGQPASISCRSSQSIVHR |
| (hu-VL1) | | NGNTYLEWFQLRPGQSPRLLIYKVSNRFSGVP |
| Light chain variant 1 | | DRFSGSGSGTDFTLKISRVEAEDVGVYYCFQ |
| (IGKV2-30*01 | | GSHVPYTFGGGTKLEIK |
| germline) | | |
| Humanised VL2 | 8 | DVVMTQSPLSLPVTLGQPASISCRSSQSIVHR |
| (hu-VL2) | | NGNTYLEWFQQRPGQSPRLLIYKVSNRFSGV |
| Light chain variant 2 | | PDRFSGSGSGTDFTLKISRVEAEDVGVYYCFQ |
| (IGKV2-30*01 | | GSHVPYTFGGGTKLEIK |
| germline) | | |
| Humanised VL3 | 9 | DVVMTQSPDSLAVSLGERATINCKSSQSIVHR |
| (hu-VL3) | | NGNTYLEWYQLKPGQPPKLLIYKVSNRFSGVP |
| Light chain variant 3 | | DRFSGSGSGTDFTLTISSLQAEDVAVYYCFQG |
| (IGKV4-1*01 | | SHVPYTFGGGTKLEIK |
| germline) | | |
| Humanised VL4 | 10 | DVVMTQSPDSLAVSLGERATINCKSSQSIVHR |
| (hu-VL4) | | NGNTYLEWYQQKPGQPPKLLIYKVSNRFSGV |
| Light chain variant 4 | | PDRFSGSGSGTDFTLTISSLQAEDVAVYYCFQ |
| (IGKV4-1*01 | | GSHVPYTFGGGTKLEIK |
| germline) | | |
| FLAG tag | 11 | DYKD |
| CDR H1 parental, | 12 | GYSFTTYT |
| VH1, VH2, VH3 and | | |
| VH4 (IMGT) | | |
| CDR H2 parental, | 13 | INPSSGYA |
| VH1, VH2, VH3 and | | |
| VH4 (IMGT) | | |
| CDR H3 parental, | 14 | AREKFYGYDY |
| VH1, VH2, VH3 and | | |
| VH4 (IMGT) | | |
| FR H1 VH1 and | 15 | QVQLVQSGAEVKKPGASVKVSCKAS |
| VH2 (IMGT and | | |
| Chothia) | | |
| FR H2 VH1 (IMGT) | 16 | IHWVRQAPGQGLEWIGY |
| FR H2 VH2 (IMGT) | 17 | MHWVRQAPGQGLEWMGY |
| FR H3 VH1 and | 18 | AYNQKFQGRVTLTADKSTSTAYMELSSLRSED |
| VH3 (IMGT) | | TAVYYC |
| FR H3 VH2 (IMGT) | 19 | AYNQKFQGRVTMTADKSTSTAYMELSSLRSE |
| | | DTAVYYC |
| FR H4 VH1, VH2, | 20 | WGQGTTVTVSS |
| VH3 and VH4 | | |
| (IMGT, Kabat and | | |
| Chothia) | | |
| CDR L1 parental, | 21 | QSIVHRNGNTY |
| VL1, VL2, VL3 and | | |
| VL4 (IMGT) | | |
| CDR L2 parental, | 22 | KVS |
| VL1, VL2, VL3 and | | |
| VL4 (IMGT) | | |
| CDR L3 parental, | 23 | FQGSHVPYT |
| VL1, VL2, VL3 and | | |
| VL4 (IMGT, Kabat | | |
| and Chothia) | | |
| FR L1 VL3 and VL4 | 24 | DVVMTQSPDSLAVSLGERATINCKSS |
| (IMGT) | | |
| FR L2 VL4 (IMGT) | 25 | LEWYQQKPGQPPKLLIY |
| FR L2 VL3 (IMGT) | 26 | LEWYQLKPGQPPKLLIY |
| FR L3 VL3 and VL4 | 27 | NRFSGVPDRFSGSGSGTDFTLTISSLQAEDVA |
| (IMGT) | | VYYC |
| FR L4 parental, | 28 | FGGGTKLEIK |
| VL1, VL2, VL3 and | | |
| VL4 (IMGT, Kabat | | |
| and Chothia) | | |
| FLAG tag deca- | 29 | GDYKDDDDKG |
| peptide | | |
| FLAG tag | 30 | DYKDDDDK |
| octapeptide | | |
| FLAG tag | 31 | MDYKDDDDK |
| nonapeptide | | |
| FLAG tag | 32 | DFKDDDK |
| heptapeptide | | |
| FLAG tag | 33 | DYKAFDNL |
| alternative | | |
| octapeptide | | |
| FLAG tag | 34 | DYKDHDG |
| alternative | | |
| heptapeptide | | |
| FLAG tag | 35 | MDFKDDDDK |
| alternative | | |
| FLAG tag | 36 | MDYKAFDNL |
| alternative | | |
| FLAG tag | 37 | DYKDHDI |
| alternative | | |
| FLAG tag | 38 | DYKDH |
| alternative | | |
| FLAG tag | 39 | DYKDD |
| alternative | | |
| FLAG tag | 40 | DYKDHD |
| alternative | | |
| FLAG tag | 41 | DYKDDD |
| alternative | | |
| CDR H1 parental, | 42 | TYTIH |
| VH1 and VH3 | | |
| (Kabat) | | |
| CDR H1 VH2 | 43 | TYTMH |
| (Kabat) | | |
| CDR H2 VH1 and | 44 | YINPSSGYAAYNQKFQG |
| VH2 (Kabat) | | |
| CDR H3 parental, | 45 | EKFYGYDY |
| VH1, VH2, VH3 and | | |
| VH4 (Kabat and | | |
| Chothia) | | |
| FR H1 VH1 and | 46 | QVQLVQSGAEVKKPGASVKVSCKASGYSF |
| VH2 (Kabat) | | |
| FR H2 VH1 and | 47 | WVRQAPGQGLEWIG |
| VH3 (Kabat) | | |
| FR H2 VH2 and | 48 | WVRQAPGQGLEWMG |
| VH4 (Kabat) | | |
| FR H3 VH1 (Kabat) | 49 | RVTLTADKSTSTAYMELSSLRSEDTAVYYCAR |
| FR H3 VH2 (Kabat) | 50 | RVTMTADKSTSTAYMELSSLRSEDTAVYYCAR |
| CDR L1 VL3 and | 51 | KSSQSIVHRNGNTYLE |
| VL4 (Kabat and | | |
| Chothia) | | |
| CDR L2 parental, | 52 | KVSNRFS |
| VL1, VL2, VL3 and | | |
| VL4 (Kabat and | | |
| Chothia) | | |
| FR L1 VL3 and VL4 | 53 | DVVMTQSPDSLAVSLGERATINC |
| (Kabat and Chothia) | | |
| FR L2 VL4 (Kabat | 54 | WYQQKPGQPPKLLIY |
| and Chothia) | | |
| FR L2 VL3 (Kabat | 55 | WYQLKPGQPPKLLIY |
| and Chothia) | | |
| FR L3 VL3 and VL4 | 56 | GVPDRFSGSGSGTDFTLTISSLQAEDVAVYYC |
| (Kabat and Chothia) | | |
| CDR H1 parental, | 57 | GYSFTTY |
| VH1, VH2, VH3 and | | |
| VH4 (Chothia) | | |
| CDR H2 parental, | 58 | NPSSGY |
| VH1, VH2, VH3 and | | |
| VH4 (Chothia) | | |
| FR H2 VH1 and | 59 | TIHWVRQAPGQGLEWIGYI |
| VH3 (Chothia) | | |
| FR H2 VH2 | 60 | TMHWVRQAPGQGLEWMGYI |
| (Chothia) | | |
| FR H3 VH1 | 61 | AAYNQKFQGRVTLTADKSTSTAYMELSSLRSE |
| (Chothia) | | DTAVYYCAR |
| FR H3 VH2 | 62 | AAYNQKFQGRVTMTADKSTSTAYMELSSLRS |
| (Chothia) | | EDTAVYYCAR |
| CDR H2 parental | 63 | YINPSSGYAAYNQNFKD |
| (Kabat) | | |
| CDR L1 parental | 64 | RSSQSIVHRNGNTYLE |
| VL1 and VL2 (Kabat | | |
| and Chothia) | | |
| FR H1 parental | 65 | QVQLQQSAGELARPGASVKMSCKAS |
| (IMGT) | | |
| FR H2 parental | 66 | IHWVKQRPGQGLEWIGY |
| (IMGT) | | |
| FR H3 parental | 67 | AYNQNFKDETTLTADKSSSTAYMQLSSLTSED |
| (IMGT) | | SAVYYC |
| FR H4 parental | 68 | WGQGTTLTVSS |
| (IMGT, Kabat and | | |
| Chothia) | | |
| FR L1 parental and | 69 | DVLMTQLPLSLPVSLGDQASISCRSS |
| VL1 (IMGT) | | |
| FR L2 parental and | 70 | LEWYLLKPGQSPKLLIY |
| VL1 (IMGT) | | |
| FR L3 parental and | 71 | NRFSGVPDRFSGSGSGTDFTLKISRVEAEDLG |
| VL1 (IMGT) | | VYYC |
| FR H1 parental | 72 | QVQLQQSAGELARPGASVKMSCKASGYSFT |
| (Kabat) | | |
| FR H2 parental | 73 | WVKQRPGQGLEWIG |
| (Kabat) | | |
| FR H3 parental | 74 | ETTLTADKSSSTAYMQLSSLTSEDSAVYYCAR |
| (Kabat) | | |
| FR L1 parental and | 75 | DVLMTQLPLSLPVSLGDQASISC |
| VL1 (Kabat and | | |
| Chothia) | | |
| FR L2 parental and | 76 | WYLLKPGQSPKLLIY |
| VL1 (Kabat and | | |
| Chothia) | | |
| FR L3 parental and | 77 | GVPDRFSGSGSGTDFTLKISRVEAEDLGVYYC |
| VL1 (Kabat and | | F |
| Chothia) | | |
| FR H2 parental | 78 | TIHWVKQRPGQGLEWIGYI |
| (Chothia) | | |
| FR H3 parental | 79 | AAYNQNFKDETTLTADKSSSTAYMQLSSLTSE |
| (Chothia) | | DSAVYYCAR |
| FR H1 VH3 and | 80 | QVQLVQSGSELKKPGASVKVSCKAS |
| VH4 (IMGT) | | |
| FR H2 VH4 (IMGT) | 81 | MNWVRQAPGQGLEWMGY |
| FR H3 VH3 (IMGT) | 82 | AYNQGFTGRFVLSADKSVSTAYLQISSLKAED |
| | | TAVYYC |
| FR H3 VH4 (IMGT) | 83 | AYNQGFTGRFVFSADKSVSTAYLQISSLKAED |
| | | TAVYYC |
| FR L1 VL2 (IMGT) | 84 | DVVMTQSPLSLPVTLGQPASISCRSS |
| FR L2 VL2 (IMGT) | 85 | LEWFQQRPGQSPRLLIY |
| FR L3 VL2 (IMGT) | 86 | NRFSGVPDRFSGSGSGTDFTLKISRVEAEDVG |
| | | VYYC |
| CDR H1 VH4 | 87 | TYTMN |
| (Kabat) | | |
| CDR H2 VH3 and | 88 | YINPSSGYAAYNQGFTG |
| VH4 (Kabat) | | |
| FR H1 VH3 and | 89 | QVQLVQSGSELKKPGASVKVSCKASGYSFT |
| VH4 (Kabat) | | |
| FR H3 VH3 (Kabat) | 90 | RFVLSADKSVSTAYLQISSLKAEDTAVYYCAR |
| FR H3 VH4 (Kabat) | 91 | RFVFSADKSVSTAYLQISSLKAEDTAVYYCAR |
| FR L1 VL2 (Kabat) | 92 | DVVMTQSPLSLPVTLGQPASISC |
| FR L2 VL2 (Kabat) | 93 | WFQQRPGQSPRLLIY |
| FR L3 VL2 (Kabat) | 94 | GVPDRFSGSGSGTDFTLKISRVEAEDVGVYYC |
| FR H1 VH3 and | 95 | QVQLVQSGSELKKPGASVKVSCKAS |
| VH4 Chothia) | | |
| FR H2 VH4 | 96 | TMNWVRQAPGQGLEWMGYI |
| (Chothia) | | |
| FR H3 VH3 | 97 | AAYNQGFTGRFVLSADKSVSTAYLQISSLKAE |
| (Chothia) | | DTAVYYCAR |
| FR H3 VH4 | 98 | AAYNQGFTGRFVFSADKSVSTAYLQISSLKAE |
| (Chothia) | | DTAVYYCAR |
| FR L1 VL2 | 99 | DVVMTQSPLSLPVTLGQPASISC |
| (Chothia) | | |
| FR L2 VL2 | 100 | WFQQRPGQSPRLLIY |
| (Chothia) | | |
| FR L3 VL2 | 101 | GVPDRFSGSGSGTDFTLKISRVEAEDVGVYYC |
| (Chothia) | | |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed antigen binding proteins, for example antibodies, that bind to FLAG tags. The antigen binding proteins as described herein are for binding to one or more FLAG tags present on any of: a recombinant protein; a recombinant peptide, a genetically modified cell (including genetically modified cells modified to express receptors on their surface, wherein the receptor includes one or more FLAG tags). In particularly preferred embodiments, the antigen binding proteins may be used for binding to one or more FLAG tags present on a chimeric antigen receptor (CAR) expressed by a genetically modified cell such as a cytotoxic cell.

The antigen binding proteins of the invention are humanised antibodies, making them particularly suitable for binding to CARs or other receptors or proteins, expressed by genetically engineered cells for use in human therapeutics. In particular, proteins that are engineered to contain a FLAG tag, such as the tags described herein, can be bound by an antigen binding protein of the invention in vitro or in vivo.

### GENERAL

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### SELECTED DEFINITIONS

As used herein, a FLAG-tag, or FLAG octapeptide, or FLAG epitope, is a polypeptide protein tag that can be added to a protein using recombinant DNA technology, having the sequence motif DYKDDDDK (SEQ ID NO: 29). The FLAG tag can be fused to the C-terminus or the N-terminus of a protein, or inserted within a protein. As such, the antigen binding proteins of the invention are capable of specifically binding to a FLAG tag, whether it is present at the C terminus or N terminus of a protein, or inserted within a protein. In specific examples, the FLAG tag can be included in a chimeric antigen receptor (CAR) construct and the antigen binding proteins of the invention are therefore capable of binding to the CAR by way of the FLAG tag.

As used herein, a “chimeric antigen receptor (CAR)” means a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding protein” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (~50 to 70 kD) covalently linked and two light chains (~23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

The FLAG tag according to the present invention is an amino-acid-based marker, as described for example in EP 0 150 126, U.S. Pat. Nos. 4,703,004, 4,782,137 and U.S. Pat. No. 4,8151,341 and which in particular contains or consists of the sequence DYK, preferably the sequence DYKD (SEQ ID NO: 11). In addition to these sequences, other amino acids can be present, preferably hydrophilic amino acids for example R (Arg), D (Asp), E (Glu) and K (Lys) and/or amino acids with aromatic side chains for example Y (Tyr), F (Phe), H (His) and W (Trp). Examples of such FLAG-tags are disclosed in the aforementioned patent specifications and can be used within the scope of the present invention.

As used herein, the term “subject” shall be taken to mean any animal including humans, for example a mammal. Exemplary subjects include but are not limited to humans and non-human primates. For example, the subject is a human.

“Antibodies” or “immunoglobulins” or “Igs” are gamma globulin proteins that are found in blood, or other bodily fluids of vertebrates that function in the immune system to bind antigen, hence identifying and neutralizing foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (VH) followed by three constant domains (CH) for each of the α and γ chains and four CH domains for μ and ε isotypes. Each L chain has at the N-terminus, a variable domain (V L) followed by a constant domain (CL) at its other end. The VL is aligned with the VH and the CL is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion which comprises the carboxy-terminal portions of both H chains held together by disulfides. The effector functions of antibodies such as ADCC are determined by sequences in the Fc region, which region is also the part recognized by Fc receptors (FcR) found on certain types of cells.

The pairing of a VH and VL together forms a “variable region” or “variable domain” including the amino-terminal domains of the heavy or light chain of the antibody. The variable domain of the heavy chain may be referred to as “VH.” The variable domain of the light chain may be referred to as “VL.” The V domain contains an antigen binding protein which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each 9-12 amino acids long. The FRs largely adopt a β-sheet configuration and the hypervariable regions form loops connecting, and in some cases forming part of, the β-sheet structure.

“Hypervariable region”, “HVR”, or “HV” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196:901-917, 1987; Chothia et al., Nature 342:877-883, 1989; and/or Al-Lazikani et al., J. Mol. Biol. 273:927-948, 1997; the numbering system of Honnegher and Plükthun J. Mol. Biol. 309:657-670, 2001; or the IMGT system discussed in Giudicelli et al., Nucleic Acids Res. 25:206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9:133-139, 1995 established that the five C-terminal amino acids of heavy chain CDR2 are not generally involved in antigen binding.

“Framework” or “FR” residues are those variable domain residues other than the hypervariable region or CDR residues herein defined. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

“A peptide for forming an antigen binding protein” generally refers to a peptide that may form a conformation that confers the specificity of an antibody for antigen. Examples include whole antibody or whole antibody related structures, whole antibody fragments including a variable domain, variable domains and fragments thereof, including light and heavy chains, or fragments of light and heavy chains that include some but not all of hypervariable regions or constant regions.

An “intact” or “whole” antibody is one which comprises an antigen-binding protein as well as a CL and at least heavy chain constant domains, CH1, CH2 and CH3. The constant domains may be native sequence constant domains (e.g. human native sequence constant domains) or amino acid sequence variant thereof.

“Whole antibody related structures” include multimerized forms of whole antibody.

“Whole antibody fragments including a variable domain” include Fab, Fab′, F(ab′)2, and Fv fragments; diabodies; linear antibodies, single-chain antibody molecules; and multispecific antibodies formed from antibody fragments.

The Fab fragment consists of an entire L chain along with the variable region domain of the H chain (VH), and the first constant domain of one heavy chain (CHI). Each Fab fragment is monovalent with respect to antigen binding, i.e., it has a single antigen-binding protein.

A Fab′ fragment differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A F(ab′)2 fragment roughly corresponds to two disulfide linked Fab fragments having divalent antigen-binding activity and is still capable of cross-linking antigen.

An “Fv” is an antibody fragment which contains a complete antigen-recognition and—binding site. This fragment consists of a dimer of one heavy- and one light-chain variable region domain in tight, non-covalent association.

In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected to form a single polypeptide chain. Preferably, the scFv polypeptide further comprises a polypeptide linker between the VH and VL domains which enables the scFv to form the desired structure for antigen binding.

A “single variable domain” is half of an Fv (comprising only three CDRs specific for an antigen) that has the ability to recognize and bind antigen, although at a lower affinity than the entire binding site.

“Diabodies” refers to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments (see preceding paragraph) with short linkers (about 5-10 residues) between the VH and VL domains such that interchain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the VH and VL domains of the two antibodies are present on different polypeptide chains. Triabodies and tetrabodies are also generally known in the art.

An “isolated antibody” is one which has been identified and separated and/or recovered from a component of its pre-existing environment. Contaminant components are materials that would interfere with therapeutic uses for the antibody, and may include enzymes, hormones, and other proteinaceous or nonproteinaceous solutes.

A “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology, or may be made using recombinant DNA methods in bacterial, eukaryotic animal or plant cells. The “monoclonal antibodies” may also be isolated from phage antibody libraries.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g. Old World Monkey, Ape etc), and human constant region sequences.

The term “anti-FLAG antibody” or “an antibody that binds to FLAG” refers to an antibody that is capable of binding FLAG tag (eg as defined herein in SEQ ID NO: 11 or 29) with sufficient affinity such that the antibody is useful as a diagnostic and/or therapeutic agent in targeting proteins or cells expressing are presenting the FLAG tag. Preferably, the extent of binding of an anti-FLAG antibody to an unrelated tag or protein is less than about 10% of the binding of the antibody to FLAG as measured, e.g., by a radioimmunoassay (RIA). In certain embodiments, an antibody that binds to FLAG has a dissociation constant (K_{D}) of <1 μM, <100 nM, <10 nM, <1 nM, or <0.1 nM.

“Binding affinity” generally refers to the strength of the sum total of noncovalent interactions between a single binding site of a molecule (e.g., an antibody) and its binding partner (e.g., an antigen). Generally, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells.

As used herein, the term “does not detectably bind” shall be understood to mean that an antigen binding protein, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilized and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore) in which the antigen binding protein is immobilized and contacted with an antigen.

An “affinity matured” antibody is one with one or more alterations in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art.

“ADCC” refers to a process called antibody-dependent cellular cytotoxicity, which is an immune response mediated primarily by natural killer (NK) cells in humans. In ADCC, FcγRIII on the surface of an NK cell recognizes the Fc region of antibody that is bound to antigen displayed on the surface of a target cell. This activates the NK cell, which releases perforins and granzymes, leading to lysis and apoptosis of the target cells.

“CDC” refers to a complex process called complement-dependent cytotoxicity that can lead to cell killing through the action of a cascade of proteins that can act through either of two major pathways.

“ADCP” refers to a process called antibody dependent cell-mediated phagocytosis. In this Fc receptor-mediated process, target cells to which antibodies are bound are engulfed by phagocytic cells, such as macrophage, monocytes, neutrophils, and dendritic cells. Multiple Fc receptors are involved in this process.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

As meant herein, an “Fc region” is a dimer consisting of two polypeptide chains joined by one or more disulfide bonds, each chain comprising part or all of a hinge domain plus a CH2 and a CH3 domain. Each of the polypeptide chains is referred to as an “Fc polypeptide chain.” To distinguish the two Fe polypeptide chains, one is referred to herein as an “A chain” and the other is referred to as a “B chain.” More specifically, the Fc regions contemplated for use with the present invention are IgG Fc regions, which can be mammalian or human IgG1, IgG2, IgG3, or IgG4 Fc regions. Among human IgG1 Fc regions, at least two allelic types are known.

An “Fc-containing protein,” as meant herein, is a protein comprising an Fc region as described herein and a binding region that binds to a target molecule. The term “Fc containing protein” encompasses an antibody or an Fc fusion protein that contains an Fc region.

The phrase “therapeutically effective amount” generally refers to an amount of an antigen binding protein of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

The words “treat” or “treatment” refer to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder. The success or otherwise of treatment may be monitored by, amongst other things, physical examination of the individual, cytopathological, serological DNA, or mRNA detection techniques.

The words “prevent” and “prevention” generally refer to prophylactic or preventative measures for protecting or precluding an individual not having a given disease or disorder from progressing to that disease or disorder.

The phrase “pharmaceutically acceptable” indicates that the substance or composition must be compatible chemically and/or toxicologically, with the other ingredients comprising a formulation, and/or the mammal being treated therewith.

### Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a VH or VL as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding protein described herein. A “moderate stringency” is defined herein as being a hybridization and/or washing carried out in 2×SSC buffer, 0.1% (w/v) SDS at a temperature in the range 45 °C to 65 °C, or equivalent conditions. A “high stringency” is defined herein as being a hybridization and/or wash carried out in 0.1×SSC buffer, 0.1% (w/v) SDS, or lower salt concentration, and at a temperature of at least 65 °C, or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tₘ) are known in the art. A temperature that is similar to (e.g., within 5 °C or within 10 °C) or equal to the Tₘ of a nucleic acid is considered to be high stringency. Medium stringency is to be considered to be within 10 °C to 20 °C or 10 °C to 15 °C of the calculated Tₘ of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157:105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525:309-322, 2009) or RNA (Kopsidas et al., Immunol. Lett. 107:163-168, 2006; Kopsidas et al. BMC Biotechnology, 7:18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370:389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

As used herein, the properties of amino acids are defined in the following table:

| Amino Acid | 3-letter code | 1-letter code | Properties |
|---|---|---|---|
| Alanine | Ala | A | aliphatic |
| | | | hydrophobic |
| | | | neutral |
| Arginine | Arg | R | polar |
| | | | hydrophilic |
| | | | charged (+) |
| Asparagine | Asn | N | polar |
| | | | hydrophilic |
| | | | neutral |
| Aspartate | Asp | D | polar |
| | | | hydrophilic |
| | | | charged (−) |
| Cysteine | Cys | C | polar |
| | | | hydrophobic |
| | | | neutral |
| Glutamine | Gln | Q | polar |
| | | | hydrophilic |
| | | | neutral |
| Glutamate | Glu | E | polar |
| | | | hydrophilic |
| | | | charged (−) |
| Glycine | Gly | G | aliphatic |
| | | | neutral |
| Histidine | His | H | aromatic |
| | | | polar |
| | | | hydrophilic |
| | | | charged (+) |
| Isoleucine | Ile | I | aliphatic |
| | | | hydrophobic |
| | | | neutral |
| Leucine | Leu | L | aliphatic |
| | | | hydrophobic |
| | | | neutral |
| Lysine | Lys | K | polar |
| | | | hydrophilic |
| | | | charged (+) |
| Methionine | Met | M | hydrophobic |
| | | | neutral |
| Phenylalanine | Phe | F | aromatic |
| | | | hydrophobic |
| | | | neutral |
| Proline | Pro | P | hydrophobic |
| | | | neutral |
| Serine | Ser | S | polar |
| | | | hydrophilic |
| | | | neutral |
| Threonine | Thr | T | polar |
| | | | hydrophilic |
| | | | neutral |
| Tryptophan | Trp | W | aromatic |
| | | | hydrophobic |
| | | | neutral |
| Tyrosine | Tyr | Y | aromatic |
| | | | polar |
| | | | hydrophobic |
| Valine | Val | V | aliphatic |
| | | | hydrophobic |
| | | | neutral |

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al.,

Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276 (9): 6591-604, 2001). Additional examples of non-immunostimulatory Fc regions are described, for example, in Dall'Acqua et al., J Immunol. 177:1129-1138 2006; and/or Hezareh J Virol; 75:12161-12168, 2001).

In another example, the Fc region is a chimeric Fc region, e.g., comprising at least one CH2 domain from an IgG4 antibody and at least one CH3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Antibody Binding Domain Containing Proteins

In another embodiment there is provided an antigen binding protein as described above wherein an amino acid sequence forming one or more of FR1, CDR1, FR2, CDR2, FR3, CDR3 and FR4 is derived from a human sequence or in the form of a human sequence.

The antigen binding protein may be presented in a humanized form including non-human (e.g., murine) and human immunoglobulin sequences. Typically, all but the CDR sequences of the antigen binding protein are from a non-human species such as mouse, rat or rabbit. In some instances, framework residues of the antigen binding protein may also be non-human. Where the antigen binding protein is provided in the form of a whole antibody, typically at least a portion of an immunoglobulin constant region (Fc) is human, thereby allowing various human effector functions.

Methods for humanizing non-human antigen binding proteins are well known in the art, examples of suitable processes including those in Jones et al., (1986) Nature, 321:522; Riechmann et al., (1988) Nature, 332:323; Verhoeyen et al., (1988) Science, 239:1534.

Phage display methods described herein using antibody libraries derived from human immunoglobulin sequences are useful for generating human antigen binding proteins and human antibodies.

Also, transgenic mammals that are incapable of expressing functional endogenous immunoglobulins, but which can express human immunoglobulin genes can be used. These mice may be generated by random or targeted insertion of the human heavy and light chain immunoglobulin genes into embryonic stem cells. The host heavy and light chain immunoglobulin genes may be rendered non-functional by the insertion or by some other recombination event, for example by homozygous deletion of the host JH region. The transfected embryonic stem cells are expanded and microinjected into blastocysts to produce chimeric mice that are then bred to produce homozygous offspring that express human antigen binding proteins. After immunization with a FLAG tag, human monoclonal antibodies can be obtained. One benefit of transgenic animal systems is that it is possible to produce therapeutically useful isotypes because the human immunoglobulin transgenes rearrange during B-cell differentiation and subsequently undergo class switching and somatic mutation in the transgenic mice.

Variable domains including CDRs and FRs of the invention may have been made less immunogenic by replacing surface-exposed residues so as to make the antibody appear as self to the immune system. Padlan, E. A., 1991, Mol. Immunol. 28, 489 provides an exemplary method. Generally, affinity is preserved because the internal packing of amino acid residues in the vicinity of the antigen binding protein remains unchanged and generally CDR residues or adjacent residues which influence binding characteristics are not to be substituted in these processes.

In another embodiment there is provided an anti-FLAG tag binding protein, immunoglobulin variable domain, antibody, dab, scFv, Fab, Fab′, F(ab′)2, Fv fragment, diabody, triabody, linear antibody, single-chain antibody molecule, or multispecific antibody as described herein, preferably with a sequence as shown in Table 1.

Lower molecular weight antibody fragments, as compared with whole antibodies may have improved access to solid tumors and more rapid clearance which may be particularly useful in therapeutic and in vivo diagnostic applications.

In certain embodiments, the antigen binding protein is provided in the form of a single chain Fv fragment (scFv). Fv and scFv are suitable for reduced nonspecific binding during in vivo use as they have intact combining sites that are devoid of constant regions. Fusion proteins including scFv may be constructed to yield fusion of an effector protein at either the amino or the carboxy terminus of an scFv.

In another embodiment there is provided a diabody or triabody or other multispecific antibody including an antigen binding protein as described above. Multispecific antibodies may be assembled using polypeptide domains that allow for multimerization. Examples include the CH2 and CH3 regions of the Fc and the CH1 and Ckappa/lambda regions. Other naturally occurring protein multimerization domains may be used including leucine zipper domain (bZIP), helix-loop-helix motif, Src homology domain (SH2, SH3), an EF hand, a phosphotyrosine binding (PTB) domain, or other domains known in the art.

In another embodiment there is provided a fusion domain or heterologous protein including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody as described herein.

A heterologous polypeptide may be recombinantly fused or chemically conjugated to an N- or C-terminus of an antigen binding protein or molecule containing same of the invention.

The heterologous polypeptide to which the antibody or antigen binding protein is fused may be useful for binding to FLAG tag present on recombinant agents, including (but not limited to): recombinant proteins comprising a FLAG tag, therapeutic antibodies or other antigen binding domain comprising proteins comprising a FLAG tag; cellular immunotherapeutics that have been genetically modified to express a FLAG tag or to express a receptor or other protein that comprises a FLAG tag. In certain examples, the antigen binding protein of the present invention may be useful for binding to chimeric antigen receptors (CARs) present on cytotoxic immune cells (such as on CAR T cells).

Further, the antigen binding protein, immunoglobulin variable domain, antibody, dab, scFv, Fab, Fab′, F(ab′)2, Fv fragment, diabody, triabody, linear antibody, single-chain antibody molecule, or multispecific antibody of the invention may be modified by glycosylation, acetylation, pegylation, phosphorylation, amidation, derivatization by known protecting/blocking groups, proteolytic cleavage, linkage to a cellular ligand or other protein, etc.

### Indirect CARs and Related Chimeric Receptors

In still further embodiments, a heterologous polypeptide to which the antibody or antigen binding protein of the invention is fused may be included as a component of a chimeric antigen protein (CAR) or variant T cell receptor. In such embodiments it will be appreciated that the CAR or variant T cell receptor that is generated comprising the antigen binding protein of the invention may be referred to as an “indirect CAR” (sometimes also referred to as a universal CAR). In an indirect CAR system, the antigen binding domain of the CAR does not directly bind to a target antigen on a target cell, but instead binds to an intermediate, wherein the intermediate comprises an antigen binding domain for directly binding the target cell. Examples of a CAR that recognise cells by way of an intermediate are known in the art, for example European patent application EP 2651442.

The skilled person will familiar with the general architecture of CARs and other modified receptors in which the antigen binding protein of the invention may be included. Typically, a “Chimeric Antigen Receptor” refers to a recombinant polypeptide construct comprising at least an extracellular antigen binding moiety, a transmembrane domain and a cytoplasmic signalling domain (also referred to herein as “an intracellular signalling domain”).

Chimeric antigen receptors are artificially constructed proteins that upon expression on the surface of a cell can induce an antigen-specific cellular response. A CAR includes at least two domains; the first domain being an antigen binding domain that binds to an antigen, or more specifically an epitope portion, or portions, of an antigen; and the second domain being a signalling domain that is capable of inducing, or participating in the induction, of an intracellular signalling pathway.

The combination of these two domains determines the antigen specificity of the CAR and the ability of the CAR to induce a desired cellular response, the latter of which is also dependent on the host cell of the CAR. For example, the activation of a CAR expressed in a T-helper cell and having a signalling domain comprising a CD3 activation domain, may, once activated by encountering its cognate antigen, induce the CD4+T-helper cell to secrete a range of cytokines. In a further example, the same CAR when expressed in a CD8+ cytotoxic T cell, once activated by a cell expressing the cognate antigen, may induce the release of cytotoxins that ultimately lead to the induction of apoptosis of the antigen-expressing cell.

In addition to the antigen-recognition domain and the signalling domain a CAR may further include additional components, or portions. For example, the CAR may include a transmembrane domain that may comprise a portion of, or may be associated with, the signalling domain of the CAR. The transmembrane domain is typically one or more hydrophobic helices, which spans the lipid bilayer of a cell and embeds the CAR within the cell membrane. The transmembrane domain of the CAR can be one determinant in the expression pattern of the CAR when associated with a cell. For example, using a transmembrane domain associated with a CD3 co-receptor can permit expression of the CAR in naïve T cells, whilst use of a transmembrane domain from a CD4 co-receptor may direct expression of a CAR in T-helper cells but not cytotoxic T cells.

A further component or portion of a CAR may be a linker domain. The linker domain (also known as the spacer or hinge domain) may span from the extracellular side of the transmembrane domain to the antigen-binding domain, thereby linking the antigen binding domain to the transmembrane domain. Whilst in some cases a linker domain is not required for a functional CAR (i.e. the antigen-recognition domain can be connected directly to the transmembrane domain) in some circumstances the use of a linker domain allows for greater efficacy of the CAR. The linker domain can have a variety of functions including allowing flexibility of the CAR to permit the necessary orientation of the antigen binding domain of the CAR for binding to an antigen. Consequently, the linker domain can be any amino acid sequence that performs this function. One non-limiting example of a linker domain is a domain having amino acid sequence homology to the hinge region of an IgG antibody, such as the IgG1 hinge region. Alternative examples include amino acid sequences having sequence homology to the C_{H2}-C_{H3} region of an antibody or portions of the CD3 co-receptor complex, the CD4 co-receptor or the CD8 co-receptor.

In some embodiments, a CAR or other modified receptor, may comprise an antigen binding protein as described herein, in the form of a sdAb, an scFv (including a multi-valent scFv), a Fab or another antibody-like structure.

The signalling domain of the CAR can be any suitable domain that is capable of inducing, or participating in the induction of, an intracellular signalling cascade upon activation of the CAR as a result of recognition of an antigen by the antigen-recognition domain of the CAR. The signalling domain of a CAR will be specifically chosen depending on the cellular outcome desired following activation of the CAR. Whilst there are many possible signalling domains, when used in immunotherapy and cancer therapy the signalling domains can be grouped into two general categories based on the receptor from which they are derived, namely activation receptors and co-stimulatory receptors.

Therefore, in some embodiments of the first aspect of the invention, the signalling domain includes a portion derived from an activation receptor. In some embodiments, the signalling domain includes a portion derived from a co-stimulatory receptor.

As used throughout the specification the term “portion”, when used with respect to an activation receptor or co-stimulatory receptor, relates to any segment of the receptor that includes a sequence responsible for, or involved in, the initiation/induction of an intracellular signalling cascade following interaction of the receptor with its cognate antigen or ligand. An example of the initiation/induction of an intracellular signalling cascade for the T cell receptor (TCR) via CD3 is outlined below.

Whilst not wishing to be bound by theory, the extracellular portion of the TCR largely comprises heterodimers of either the clonotypic TCRα and TCRβ chains (the TCRα/β receptor) or the TCRγ and TCRδ chains (the TCRγδ receptor). These TCR heterodimers generally lack inherent signalling transduction capabilities and therefore they are non-covalently associated with multiple signal transducing subunits of CD3 (primarily CD3-ζ, -γ, -δ, and -ε). Each of the gamma, delta, and epsilon chains of CD3 has an intracellular (cytoplasmic) portion that includes a single Immune-receptor-Tyrosine-based-Activation-Motif (ITAM), whilst the CD3-ζ chain includes three tandem ITAMs. Upon engagement of the TCR by its cognate antigen in the presence of MHC, and the association of a requisite co-receptor such as CD4 or CD8, signalling is initiated which results in a tyrosine kinase (namely Lck) phosphorylating the two tyrosine residues within the intracellular ITAM(s) of the CD3 chains. Subsequently, a second tyrosine kinase (ZAP-70—itself activated by Lck phosphorylation) is recruited to biphosphorylate the ITAMs. As a result, several downstream target proteins are activated which eventually leads to intracellular conformational changes, calcium mobilisation, and actin cytoskeleton re-arrangement that when combined ultimately lead to activation of transcription factors and induction of a T cell immune response.

As used throughout the specification the term “activation receptor” relates to receptors, or co-receptors that form a component of, or are involved in the formation of, the T cell receptor (TCR) complex, or receptors involved in the specific activation of immune cells as a result of recognition of an antigenic or other immunogenic stimuli.

Non-limiting examples of such activation receptors include components of the T cell receptor-CD3 complex (CD3-ζ, -γ, -δ, and -ε), the CD4 co-receptor, the CD8 co-receptor, Fc receptors or Natural Killer (NK) cell associated activation receptors such a LY-49 (KLRA1), natural cytotoxicity receptors (NCR, preferably NKp46, NKp44, NKp30 or NKG2 or the CD94/NKG2 heterodimer). Consequently, in some embodiments of the present invention, the signalling domain includes a portion derived from any one or more of a member of the CD3 co-receptor complex (preferably the CD3-ζ chain or a portion thereof), the CD4 co-receptor, the CD8 co-receptor, a Fc Receptor (FcR) (preferably the FcεRI or FcγRI) or NK associated receptors such a LY-49.

The specific intracellular signal transduction portion of each of the CD3 chains are known in the art, as are intracellular portions of FC receptors.

Various combinations of portions of activation receptors can be utilized to form the transmembrane (TM) and intracellular (IC) portions of the CAR for example the CD3ζ TM and CD3ζ IC (Landmeier S. et al. Cancer Res. 2007; 67:8335-43; Guest R D. et al., J Immunother. 2005, 28:203-11; Hombach A A. et al. J Immunol. 2007; 178:4650-7), the CD4 TM and CD3Z IC (James SE. et al. J Immunol. 2008; 180:7028-38), the CD8 TM and CD3ζ IC (Patel SD. Et al. Gene Ther. 1999; 6:412-9), and the FcεRIγ TM and the FcεRIγ IC (Haynes NM. et al. J Immunol. 2001; 166:182-7; Annenkov A E. et al. J Immunol. 1998; 161:6604-13).

As used throughout the specification the term “co-stimulatory receptor” relates to receptors or co-receptors that assist in the activation of an immune cell upon antigen specific inducement of an activation receptor. As will be understood, co-stimulatory receptors do not require the presence of antigen and are not antigen specific, but are typically one of two signals, the other being an activation signal, which is required for the induction of an immune cellular response. In the context of an immune response a co-stimulation receptor is typically activated by the presence of its expressed ligand on the surface of an antigen-presenting cell (APC) such as a dendritic cell or macrophage. With specific regard to T cells, co-stimulation is necessary to lead to cellular activation, proliferation, differentiation and survival (all of which are generally referred to under the umbrella of T cell activation), whilst presentation of an antigen to a T cell in the absence of co-stimulation can lead to anergy, clonal deletion and/or the development of antigen specific tolerance. Importantly, co-stimulatory molecules can inform the T cell response to a simultaneously encountered antigen. Generally, an antigen encountered in the context of a ‘positive’ co-stimulatory molecule will lead to activation of the T cell and a cellular immune response aimed at eliminating cells expressing that antigen.

Whilst an antigen encountered in the context of a ‘negative’ co-receptor will lead to an induced state of tolerance to the co-encountered antigen.

Non-limiting examples of T cell co-stimulatory receptors include CD27, CD28, CD30, CD40, DAP10, OX40, 4-1BB (CD137), ICOS. Specifically, CD27, CD28, CD30, CD40, DAP10, OX40, 4-1BB (CD137), and ICOS all represent ‘positive’ co-stimulatory molecules that enhance activation of a T cell response. Accordingly, in some embodiments of the present invention, the signalling domain includes a portion derived from any one or more of CD27, CD28, CD30, CD40, DAP10, OX40, 4-1BB (CD137) and ICOS.

In some embodiments the signalling domain includes a portion derived from the CD28, OX40 or 4-1BB co-stimulatory receptors. In some embodiments, the signalling domain includes a portion of the CD28 co-stimulatory receptor. In some embodiments, the signalling domain includes a portion of the OX40 costimulatory receptor. Various combinations of portions of co-stimulatory receptors can be utilized to form the transmembrane (TM) and intracellular (IC) portions of the CAR. For example the CD8 TM and DAP10 IC or CD8 TM and 4-1BB IC (Marin V. et al. Exp Hematol. 2007; 35:1388-97), the CD28 TM and the CD28 IC (Wilkie S. et al. J Immunol. 2008; 180:4901-9; Maher J. et al. Nat Biotechnol. 2002; 20:70-5), and the CD8 TM and the CD28 IC (Marin V. et al. Exp Hematol. 2007; 35:1388-97).

### Further Details of Antigen Binding Proteins of the Invention

Antigen binding proteins of the invention can be composed of amino acids joined to each other by peptide bonds or modified peptide bonds, i.e., peptide isosteres, and may contain amino acids other than the 20 gene-encoded amino acids. Antigen binding proteins of the invention may be modified by natural processes, such as post-translational processing, or by chemical modification techniques which are well known in the art. Such modifications are well described in basic texts, as well as in research literature. Modifications can occur anywhere in the antigen binding protein, including the peptide backbone, the amino acid side-chains and the amino or carboxyl termini, or on moieties such as carbohydrates. It will be appreciated that the same type of modification may be present in the same or varying degrees at several proteins in a given antigen binding protein. Also, a given antigen binding protein may contain many types of modifications. An antigen binding protein may be branched, for example, as a result of ubiquitination, and they may be cyclic, with or without branching. Cyclic, branched, and branched cyclic antigen binding proteins may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

In another embodiment there is provided a conjugate in the form of an antigen binding protein, immunoglobulin variable domain, antibody, Fab, dab, scFv, diabody, triabody or fusion protein as described above conjugated to a cytotoxic agent such as a chemo therapeutic agent, a drug, a growth inhibitory agent, a toxin (e.g., an enzymatically active toxin of bacterial, fungal, plant, or animal origin, or fragments thereof), or a label such as a radioactive isotope (i.e., a radio conjugate). In another aspect, the invention further provides methods of using the immunoconjugates. In one aspect, an immunoconjugate comprises any of the above variable domains covalently attached to a cytotoxic agent or a detectable agent.

In another embodiment there is provided an antibody for binding to an antigen binding protein, immunoglobulin variable domain, antibody, dab, scFv, Fab, Fab′, F (ab′)2, Fv fragment, diabody, triabody, linear antibody, single-chain antibody molecule, or multispecific antibody, fusion protein or conjugate as described above.

In another embodiment there is provided a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described above.

A polynucleotide encoding an CDR or FR according to any one of the general formulae described above, or an antigen binding protein comprised of same, may be generated from a nucleic acid from any source, for example by chemical synthesis or isolation from a cDNA or genomic library. For example, a cDNA library may be generated from an antibody producing cell such as a B cell, plasma cell or hybridoma cell and the relevant nucleic acid isolated by PCR amplification using oligonucleotides directed to the particular clone of interest. Isolated nucleic acids may then be cloned into vectors using any method known in the art. The relevant nucleotide sequence may then be mutagenized using methods known in the art e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N. Y. and Ausubel et al., eds., 1998, Current Protocols in Molecular Biology, John Wiley & Sons, NY), to generate antigen binding proteins having a different amino acid sequence, for example to create amino acid substitutions, deletions, and/or insertions.

### Protein Production

In another embodiment there is provided a method for producing an anti-FLAG antigen binding protein as described above including expressing a nucleic acid as described above in a cell or non-human animal as described above.

The production of an antigen binding protein of the invention generally requires an expression vector containing a polynucleotide that encodes the antigen binding protein of the invention. A polynucleotide encoding an antigen binding protein of the invention may be obtained and sub-cloned into a vector for the production of an antigen binding protein by recombinant DNA technology using techniques well-known in the art, including techniques described herein. Many different expression systems are contemplated including the use of mammalian cells including human cells for production and secretion of antigen binding proteins. Examples of cells include 293F, CHO and the NSO cell line.

Expression vectors containing protein coding sequences and appropriate transcriptional and translational control signals can be constructed using methods known in the art. These include in vitro recombinant DNA techniques, synthetic techniques and in vivo genetic recombination. In certain embodiments there is provided a replicable vector having a nucleic acid encoding an antigen binding protein operably linked to a promoter.

Cells transfected with an expression vector may be cultured by conventional techniques to produce an antigen binding protein. Thus, in certain embodiments, there is provided host cells or cell transfectants containing a polynucleotide encoding an antigen binding protein of the invention operably linked to a promoter. The promoter may be heterologous. A variety of host-expression vector systems may be utilized and in certain systems the transcription machinery of the vector system is particularly matched to the host cell. For example, mammalian cells such as Chinese hamster ovary cells (CHO) may be transfected with a vector including the major intermediate early gene promoter element from human cytomegalovirus. Additionally or alternatively, a host cell may be used that modulates the expression of inserted sequences, or modifies and processes the gene product as required, including various forms of post-translational modification. Examples of mammalian host cells having particular post translation modification processes include CHO, VERY, BHK, Hela, COS, MDCK, 293, 3T3, W138, BT483, Hs578T, HTB2, BT2O and T47D, NSO, CRL7030 and HsS78Bst cells.

Depending upon the use intended for the protein molecule, a number of bacterial expression vectors may be advantageously selected. In one example, vectors that cause the expression of high levels of fusion protein products that are readily purified, such as the E. coli expression vector pUR278 may be used where a large quantity of an antigen binding protein is to be produced. The expression product may be produced in the form of a fusion protein with lacZ. Other bacterial vectors include pIN vectors and the like. pGEX vectors may also be used to express foreign polypeptides as fusion proteins with glutathione-S-transferase (GST). These fusion proteins are generally soluble and can easily be purified from lysed cells by adsorption and binding to glutathione-agarose affinity matrix, followed by elution in the presence of free glutathione. A thrombin and/or factor Xa protease cleavage site may be provided in the expressed polypeptide so that the cloned target gene product can be released from the GST moiety.

Autographa californica nuclear polyhedrosis virus (AcNPV) may be used as a vector to express foreign genes in an insect system, including Spodoptera frugiperda cells. The particular promoter used may depend on where the protein coding is inserted into the sequence. For example, the sequence may be cloned individually into the polyhedrin gene and placed under control of the polyhedrin promoter.

Virus based expression systems may be utilized with mammalian cells such as an adenovirus, whereby the coding sequence of interest may be ligated to the adenoviral late promoter and tripartite leader sequence. In vitro or in vivo recombination may then be used to insert this chimeric gene into the adenoviral genome. Insertions into region E1 or E3 will result in a viable recombinant virus that is capable of expressing the antigen binding protein in infected host cells. Specific initiation signals including the ATG initiation codon and adjacent sequences may be required for efficient translation of inserted antigen binding protein coding sequences. Initiation and translational control signals and codons can be obtained from a variety of origins, both natural and synthetic. Transcription enhancer elements and transcription terminators may be used to enhance the efficiency of expression of a viral based system.

Where long-term, high-yield production of recombinant proteins is required, stable expression is preferred. Generally, a selectable marker gene is used whereby following transfection, cells are grown for 1-2 days in an enriched media and then transferred to a medium containing a selective medium in which cells containing the corresponding selectable marker, for example, antibiotic resistance can be screened. The result is that cells that have stably integrated the plasmid into their chromosomes grow and form foci that in turn can be cloned and expanded into cell lines. The herpes simplex virus thymidine kinase, hypoxanthineguanine phosphoribosyltransferase and adenine phosphoribosyltransferase genes are examples of genes that can be employed in tk-, hgprt- or aprT-cells, respectively, thereby providing appropriate selection systems. The following genes: dhfr, which confers resistance to methotrexate; gpt, which confers resistance to mycophenolic acid; neo, which confers resistance to the aminoglycoside G-418; and hygro, which confers resistance to hygromycin are examples of genes that can be used in anti-metabolite selection systems.

An antigen binding protein of the invention may be purified by a recombinant expression system by known methods including ion exchange chromatography, affinity chromatography (especially affinity for the specific antigens Protein A or Protein G) and gel filtration column chromatography), centrifugation, differential solubility, or by any other standard technique for the purification of proteins. Purification may be facilitated or assisted by providing the antigen binding protein in the form of a fusion protein.

Large quantities of the antigen binding proteins of the invention may be produced by a scalable process starting with a pilot expression system in a research laboratory that is scaled up to an analytical scale bioreactor (typically from 5 L to about 50 L bioreactors) or production scale bioreactors (for example, but not limited to 75 L, 100 L, 150 L, 300 L, or 500 L). Desirable scalable processes include those wherein there are low to undetectable levels of aggregation as measured by HPSEC or rCGE, typically no more than 5% aggregation by weight of protein down to no more than 0.5% by weight aggregation of protein. Additionally or alternatively, undetectable levels of fragmentation measured in terms of the total peak area representing the intact antigen binding protein may be desired in a scalable process so that at least 80% and as much as 99.5% or higher of the total peak area represents intact antigen binding protein. In other embodiments, the scalable process of the invention produces antigen binding proteins at production efficiency of about from 10 mg/L to about 300 mg/L or higher.

Various techniques have been developed for the production of antibody fragments including proteolytic digestion of intact antibodies and recombinant expression in host cells. With regard to the latter, as described below, Fab, Fv and scFv antibody fragments can all be expressed in and secreted from E. coli, antibody fragments can be isolated from the antibody phage libraries and Fab′-SH fragments can be directly recovered from E. coli and chemically coupled to form F(ab′)2 fragments. In another approach, F(ab′)2 fragments are isolated directly from recombinant host cell culture.

In another embodiment there is provided a vector including a nucleic acid described above. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The antigen binding site may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the antigen binding site-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader, or acid phosphatase leader or the C. albicans glucoamylase leader. In mammalian cell expression, mammalian signal sequences may be used to direct secretion of the protein, such as signal sequences from secreted polypeptides of the same or related species, as well as viral secretory leaders.

Polynucleotide sequences encoding polypeptide components of the antigen binding protein of the invention can be obtained using standard recombinant techniques as described above. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells.

Such sequences are well known for a variety of bacteria, yeast, and viruses. The origin of replication from the plasmid pBR322, which contains genes encoding ampicillin (Amp) and tetracycline (Tet) resistance, provides an easy means for identifying transformed cells, is suitable for most Gram-negative bacteria. The 2 μm plasmid origin is suitable for yeast. Various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron (a cistron being segment of DNA that contains all the information for production of single polypeptide) pairs. A promoter is an untranslated regulatory sequence located upstream (5′) to a cistron that modulates its expression. Prokaryotic promoters typically fall into two classes, inducible and constitutive. An inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems, alkaline phosphatase, a tryptophan (trp) promoter system and hybrid promoters such as the tac or the trc promoter. Promoters for use in bacterial systems also will contain a Shine-Dalgarno (S.D.) sequence operably linked to the DNA encoding an antigen binding protein of the invention. However, other promoters that are functional in bacteria (such as other known bacterial or phage promoters) are suitable as well. Their nucleotide sequences have been published, thereby enabling a skilled person operably to ligate them to cistrons encoding the target light and heavy chains using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB strains) provide cytoplasm conditions that are favourable for disulfide bond formation, thereby permitting proper folding and assembly of expressed protein subunits.

The present invention provides an expression system in which the quantitative ratio of expressed polypeptide components can be modulated in order to maximize the yield of secreted and properly assembled antigen binding proteins of the invention.

Such modulation is accomplished at least in part by simultaneously modulating translational strengths for the polypeptide components.

In terms of expression in eukaryotic host cells, the vector components generally include, but are not limited to, one or more of the following: a signal sequence, an origin of replication, one or more marker genes, an enhancer element, a promoter, and a transcription termination sequence.

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

Generally, an origin of replication component is not needed for mammalian expression vectors. For example, the SV40 origin may typically be used only because it contains the early promoter.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance, and thus survive the selection regimen. Examples of such dominant selection use the drugs neomycin, mycophenolic acid and hygromycin.

An example of suitable selectable markers for mammalian cells are those that enable the identification of cells competent to take up the antigen binding protein-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096) that is prepared and propagated. For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors usually contain a promoter operably linked to the antigen binding protein encoding nucleic acid sequence to direct mRNA synthesis. Promoters recognized by a variety of potential host cells are well known.

Eukaryotic genes generally have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes including enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Antigen binding protein transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the antigen binding protein by higher eukaryotes may be increased by inserting an enhancer sequence into the vector. Enhancer sequences include those known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antigen binding protein.

In another embodiment there is provided a cell including a vector or nucleic acid described above. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell.

Examples of prokaryotic cells are those generally used for cloning like E. coli or Bacillus subtilis. Furthermore, eukaryotic cells comprise, for example, fungal or animal cells.

Examples for suitable fungal cells are yeast cells, preferably those of the genus Saccharomyces and most preferably those of the species Saccharomyces cerevisiae.

Examples of animal cells are, for instance, insect cells, vertebrate cells, preferably mammalian cells, such as e.g. HEK293, NSO, CHO, MDCK, U2-OS, Hela, NIH3T3, MOLT-4, Jurkat, PC-12, PC-3, IMR, NT2N, Sk-n-sh, CaSki, C33A. These host cells, e.g. CHO-cells, may provide post-translational modifications to the antibody molecules of the invention, including leader peptide removal, folding and assembly of H (heavy) and L (light) chains, glycosylation of the molecule at correct sides and secretion of the functional molecule.

Further suitable cell lines known in the art are obtainable from cell line depositories, like the American Type Culture Collection (ATCC).

In another embodiment there is provided an animal including a cell described above. In certain embodiments, animals and tissues thereof containing a transgene are useful in producing the antigen binding proteins of the invention. The introduction of the nucleic acid molecules as transgenes into non-human hosts and their subsequent expression may be employed for the production of the antigen binding proteins, for example, the expression of such a transgene in the milk of the transgenic animal provide for means of obtaining the antigen binding proteins in quantitative amounts. Useful transgenes in this respect comprise the nucleic acid molecules of the invention, for example, coding sequences for the antigen binding proteins described herein, operatively linked to promoter and/or enhancer structures from a mammary gland specific gene, like casein or β-Lactoglobulin. The animal may be non-human mammals, most preferably mice, rats, sheep, calves, dogs, monkeys or apes.

### Binding to Target Antigen

Methods for determining the successful binding of an antigen binding protein of the invention to its target antigen (i.e. FLAG tag) are well known in the art. Non-limiting examples of such methods are described herein in the Examples. Methods for confirmation of the specificity and binding affinity of an antigen binding protein include use of Western blotting, ELISA, immunohistochemistry and Biacore methods, which are all within the skill set of a person skilled in the art.

### Kits

In another embodiment there is provided a kit or article of manufacture including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described above.

In other embodiments there is provided a kit for use in a use mentioned above, the kit including:
a container holding a therapeutic composition in the form of one or more of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition;
a label or package insert with instructions for use.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use.

The kit may comprise (a) a therapeutic composition; and (b) a second container with a second active principle or ingredient contained therein. The kit in this embodiment of the invention may further comprise a package insert indicating that the and other active principle can be used to treat a disorder or prevent a complication stemming from cancer. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic composition. In one embodiment, the device is a syringe. The device may hold 1-2 mL of the therapeutic composition. The therapeutic composition may be provided in the device in a state that is ready for use or in a state requiring mixing or addition of further components.

In other embodiments there is provided a kit for use in a diagnostic application mentioned above, the kit including:
a container holding a diagnostic composition in the form of one or more of an antigen binding protein, immunoglobulin variable domain, antibody, Fab, dab, scFv, diabody, triabody, fusion protein or conjugate;
a label or package insert with instructions for use.

The kit may comprise (a) a diagnostic composition; and (b) a second container with a second diagnostic agent or second label contained therein. It may further include other materials desirable from a commercial and user standpoint, including other buffers, diluents, filters etc.

It will be understood that the invention disclosed and defined in this specification extends to all alternative combinations of two or more of the individual features mentioned or evident from the text or drawings. All of these different combinations constitute various alternative aspects of the invention.

The examples that follow are intended to illustrate but in no way limit the present invention.

### EXAMPLES

### Example 1: Humanisation of Chimeric Antibody

A mouse anti-FLAG antibody was obtained. For the purpose of identifying complementarity determining regions (CDRs) and analysing the closest matching germline sequences the IMGT Domain Gap Align tool was used (Ehrenmann F., Kaas Q. and Lefranc M.-P. Nucleic Acids Res., 2010; 38: D301-D307).

Molecular models were built for VH and VL domains based on homology to previously published antibody crystal structures using in-house software. PDB files can be provided on request for viewing in any molecular visualization software. Images have been generated using PyMol (The PyMOL Molecular Graphics System, Version 2.0 Schrödinger, LLC).

Antibody sequences were analysed for specific liabilities based on published protein motifs. Analysis was performed by analysing for the following motifs where X represents any amino acid apart from Proline as described in the below table:

| Risk | Liability | Motif |
|---|---|---|
| High | Glycosylation | NXS or NXT |
| | Free Cysteine | C |
| | Deamidation | NG, NS or QG |
| | Isomerization | DG, DP or DS |
| Medium | Deamidation | NH |
| | Hydrolysis | NP |
| | Cleavage | TS, KK, RK or KR |
| | Transglutaminase | LLQG |
| | Integrin binding | RGD or KGD |
| | Plastic binding | FHENSP or WXXW |

At the start of the humanization process a homology model of the parental VH and VL is built in a single chain Fv (scFv) format. Modelling is done in 4 stages: gathering homologous sequences; fold library scanning; loop modelling; side chain placement. The resulting model is used to guide the choice of “donor” or “acceptor” amino acids during the humanization process.

The parental VH and VL sequences are aligned with a panel of human germline sequences. This panel has been filtered to select germline sequences that do not contain unwanted sequence liabilities, particularly N-linked glycosylation sites and free cysteines. The closest matching germlines from two different VH and VL families are selected. A humanization algorithm is then used to select CDR and framework amino acids to graft from the donor parental sequences onto the human acceptor germline sequence. Four VH and four VL sequences are produced, giving a possible 16 antibodies. Percentage identity to human is calculated using the IMGT Domain Gap Align tool (Ehrenmann F., Kaas Q. and Lefranc M.-P. Nucleic Acids Res., 2010; 38: D301-D307). Sequence liabilities are determined based on sequence motifs.

The VH and VL sequences were run through the IGMT Gap Align tool to analyse against all known antibody germline sequences. CDR regions were assigned using the IMGT definition. As expected, the sequence is most closely aligned to mouse, specifically the IGHV1-4*01 family for the VH and IGK1-117*01 for the VL. The closest matching germlines for the parental SEQ ID NO: 1 were germlines IGHV1-46*01, IGHV7-4-1*02, IGKV2-30*01 and IGKV4-1*01.

The below table summaries the original parental and humanized sequences:

| | | | % |
|---|---|---|---|
| | | | identity |
| | | Human | to |
| ID | Format | germline | human |
| Parental | | — | 66.3% |
| hu-VH1 | Humanized | IGHV1- | 84.7% |
| | | 46*01 | |
| hu-VH2 | Humanized | IGHV1- | 87.8% |
| | | 46*01 | |
| hu-VH3 | Humanized | IGHV7- | 82.7% |
| | | 4-1*02 | |
| hu-VH4 | Humanized | IGHV7- | 86.7% |
| | | 4-1*02 | |
| Parental | | — | 80.0% |
| hu-VL1 | Humanized | IGKV2- | 90.0% |
| | | 30*01 | |
| hu-VL2 | Humanized | IGKV2- | 91.0% |
| | | 30*01 | |
| hu-VL3 | Humanized | IGKV4- | 80.2% |
| | | 1*01 | |
| hu-VL4 | Humanized | IGKV4- | 81.2% |
| | | 1*01 | |

The combination of 4 different heavy and light chain framework regions resulted in the generation of 16 different V_{H}/V_{L} combinations.

The different VH/VL combinations of the 16 antibodies are summarised in the below table:

| Proposed antibodies | | VH name | VL name |
|---|---|---|---|
| | Parental | VH | VL |
| 1 | Humanized-v1 | hu-VH1 | hu-VL1 |
| 2 | Humanized-v2 | hu-VH1 | hu-VL2 |
| 3 | Humanized-v3 | hu-VH1 | hu-VL3 |
| 4 | Humanized-v4 | hu-VH1 | hu-VL4 |
| 5 | Humanized-v5 | hu-VH2 | hu-VL1 |
| 6 | Humanized-v6 | hu-VH2 | hu-VL2 |
| 7 | Humanized-v7 | hu-VH2 | hu-VL3 |
| 8 | Humanized-v8 | hu-VH2 | hu-VL4 |
| 9 | Humanized-v9 | hu-VH3 | hu-VL1 |
| 10 | Humanized-v10 | hu-VH3 | hu-VL2 |
| 11 | Humanized-v11 | hu-VH3 | hu-VL3 |
| 12 | Humanized-v12 | hu-VH3 | hu-VL4 |
| 13 | Humanized-v13 | hu-VH4 | hu-VL1 |
| 14 | Humanized-v14 | hu-VH4 | hu-VL2 |
| 15 | Humanized-v15 | hu-VH4 | hu-VL3 |
| 16 | Humanized-v16 | hu-VH4 | hu-VL4 |

All humanized IgG1 formats expressed well and were successfully purified for characterization by SDS-PAGE and SEC-HPLC to assess protein quality. The titre, amount of final antibody purified and the monomer content are described in the below table:

| | | | Titre | Purified | Monomer |
|---|---|---|---|---|---|
| ID | Format | % | (mg/l) | (mg) | (%) |
| Parental | IgG1 | VH: 66.3 | 10 | 0.8 | 100 |
| | | VL: 80 | | | |
| V1 | Humanized | VH: 84.7 | 22.5 | 1.8 | 99.2 |
| | human IgG1 | VL: 90.0 | | | |
| V2 | Humanized | VH: 84.7 | 1.25 | 0.1 | 100 |
| | human IgG1 | VL: 91.0 | | | |
| V3 | Humanized | VH: 84.7 | 50 | 4 | 100 |
| | human IgG1 | VL: 80.2 | | | |
| V4 | Humanized | VH: 84.7 | 50 | 4 | 99.4 |
| | human IgG1 | VL: 81.2 | | | |
| V5 | Humanized | VH: 87.8 | 24.5 | 1.4 | 98.0 |
| | human IgG1 | VL: 90.0 | | | |
| V6 | Humanized | VH: 87.8 | 8.5 | 0.7 | 97.8 |
| | human IgG1 | VL: 91.0 | | | |
| V7 | Humanized | VH: 87.8 | 47.5 | 3.8 | 98.5 |
| | human IgG1 | VL: 80.2 | | | |
| V8 | Humanized | VH: 87.8 | 40 | 3.2 | 98.5 |
| | human IgG1 | VL: 81.2 | | | |
| V9 | Humanized | VH: 82.7 | 15 | 1.2 | 99.14 |
| | human IgG1 | VL: 90.0 | | | |
| V10 | Humanized | VH: 82.7 | 10 | 0.8 | 100 |
| | human IgG1 | VL: 91.0 | | | |
| V11 | Humanized | VH: 82.7 | 52.5 | 4.2 | 99.5 |
| | human IgG1 | VL: 80.2 | | | |
| V12 | Humanized | VH: 82.7 | 49 | 3.9 | 99.7 |
| | human IgG1 | VL: 81.2 | | | |
| V13 | Humanized | VH: 86.7 | 2.5 | 0.2 | 100 |
| | human IgG1 | VL: 90.0 | | | |
| V14 | Humanized | VH: 86.7 | 2.5 | 0.2 | 99.4 |
| | human IgG1 | VL: 91.0 | | | |
| V15 | Humanized | VH: 86.7 | 27.5 | 2.2 | 99.6 |
| | human IgG1 | VL: 80.2 | | | |
| V16 | Humanized | VH: 86.7 | 37.5 | 3 | 100 |
| | human IgG1 | VL: 81.2 | | | |

### Example 2: Characterisation of Humanised Antibodies

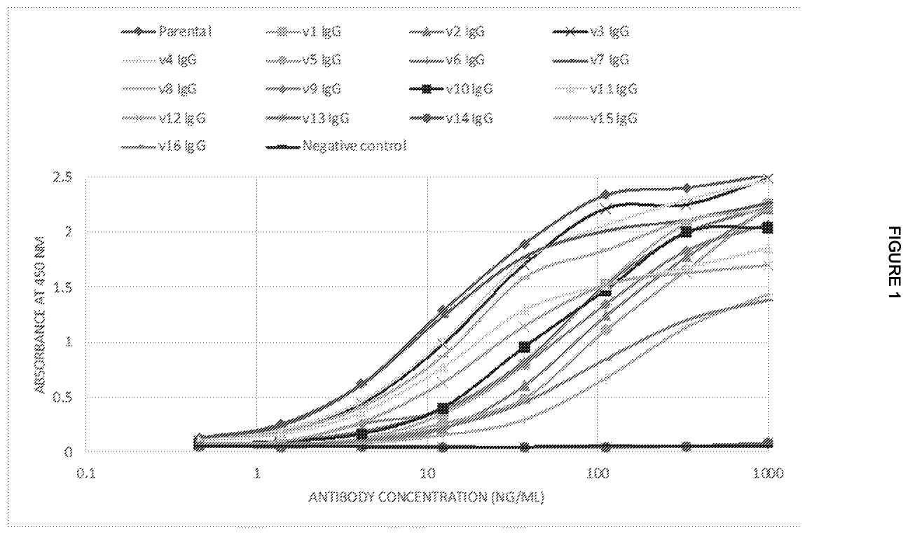

All 16 IgG format and scFv format humanised antibodies were assessed for their ability to binding to target (FLAG tag) using ELISA. Results are shown in FIG. 1.

Although most humanised variants retained the ability to bind to target, 3 humanised IgG clones (v3, v4 and v7) had greater affinity. These three IgG clones and corresponding scFV formats were selected for further analysis.

Humanized antibodies were able to demonstrate high affinity binding comparable to a parental mouse antibody (i.e. mouse VH/VL in the context of human constant regions).

The below table summarises the antibodies affinity and stoichiometry as measured by Biacore:

| Antibody | K_{D} (M) | Binding Stoichiometry |
|---|---|---|
| V4 scFv | 2.2E−08 | 2.8 |
| V3 scFv | 1.9E−08 | 5.5 |
| V7 scFv | 1.5E−08 | 3.7 |
| V7 IgG | 1.3E−09 | 1.1 |
| V3 IgG | 1.0E−09 | 1.2 |
| Parental | 9.6E−10 | 1.3 |
| V4 IgG | 9.1E−10 | 1.1 |

Four IgG antibodies (parental mouse antibody and 3 humanised antibodies: v3, v4 and v7) were compared with the corresponding humanised scFvs to a single antigen binding by Biacore. Mouse scFv was not able to be expressed and therefore not able to be used for affinity comparison. IgG antibodies bound more tightly to the antigen in comparison to scFv by almost 10-fold. This is expected as IgG is bivalent in nature and the avidity will result in tighter binding compared to monovalent expressed scFv.

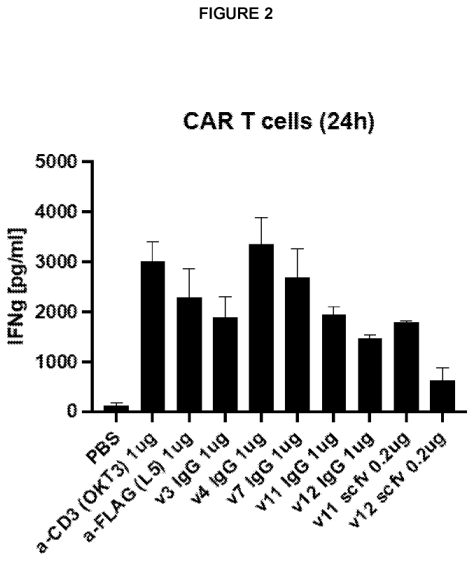

Antibodies v3, v4, v7 and v11 (in IgG format and also in scFv format in the case of v11) were further characterised. The antibodies were evaluated for their ability to activate anti-HER2-FLAG CAR T cells and induce IFN-γ. The antibodies induced IFN-γ expression as shown in FIG. 2. Antibody v11 was also included in the assessment in both IgG and scFv formats. The results indicate that antibodies v7 and v4, respectively, induced the greatest amounts of IFN-γ by CAR T cells.

Based on these results, antibody variants v4 and v7 were selected for future studies.

It will be understood that the invention disclosed and defined in this specification extends to all alternative combinations of two or more of the individual features mentioned or evident from the text or drawings. All of these different combinations constitute various alternative aspects of the invention.

1. An antigen binding protein for binding to FLAG tag, the protein comprising an antigen binding domain comprising a CDRH1, a CDRH2 and/or a CDRH3 of an antigen binding domain having a variable heavy chain (VH) as defined in any one of SEQ ID NOs: 2 to 5 and a CDRL1, a CDRL2 and/or a CDRL3 of a variable light chain (VL) as defined in any one of SEQ ID NOs: 7 to 10.

2. The antigen binding protein of claim 1, wherein the protein is capable of specifically binding to a FLAG tag or variant thereof comprising an amino acid sequence as set forth in any one of SEQ ID NOs: 11 or 29 to 41.

3. The antigen binding protein of claim 1 or 2, wherein the protein is for specifically binding to a peptide comprising or consisting of the amino acid sequence DYKDDDDK (SEQ ID NO: 30).

4. The antigen binding protein of any one of claims 1 to 3, wherein the antigen binding protein is for specifically binding to a FLAG tag comprised in or on: a recombinant or therapeutic protein, a cellular immunotherapeutic, a receptor present on a cellular immunotherapeutic.

5. The antigen binding protein of any one of claims 1 to 4, wherein the protein competitively inhibits the binding to a FLAG tag of an antibody:
comprising a VH comprising a sequence as set forth in SEQ ID NO: 1 and a VL comprising a sequence as set forth in SEQ ID NO: 6;
comprising a VH comprising a sequence as set forth in SEQ ID NO: 2 and a VL comprising a sequence as set forth in SEQ ID NO: 7;
comprising a VH comprising a sequence as set forth in SEQ ID NO: 2 and a VL comprising a sequence as set forth in SEQ ID NO: 8;
comprising a VH comprising a sequence as set forth in SEQ ID NO: 2 and a VL comprising a sequence as set forth in SEQ ID NO: 9;
comprising a VH comprising a sequence as set forth in SEQ ID NO: 2 and a VL comprising a sequence as set forth in SEQ ID NO: 10;
comprising a VH comprising a sequence as set forth in SEQ ID NO: 3 and a VL comprising a sequence as set forth in SEQ ID NO: 7;
comprising a VH comprising a sequence as set forth in SEQ ID NO: 3 and a VL comprising a sequence as set forth in SEQ ID NO: 8;
comprising a VH comprising a sequence as set forth in SEQ ID NO: 3 and a VL comprising a sequence as set forth in SEQ ID NO: 9;
comprising a VH comprising a sequence as set forth in SEQ ID NO: 3 and a VL comprising a sequence as set forth in SEQ ID NO: 10;
comprising a VH comprising a sequence as set forth in SEQ ID NO: 4 and a VL comprising a sequence as set forth in SEQ ID NO: 7;
comprising a VH comprising a sequence as set forth in SEQ ID NO: 4 and a VL comprising a sequence as set forth in SEQ ID NO: 8;
comprising a VH comprising a sequence as set forth in SEQ ID NO: 4 and a VL comprising a sequence as set forth in SEQ ID NO: 9;
comprising a VH comprising a sequence as set forth in SEQ ID NO: 4 and a VL comprising a sequence as set forth in SEQ ID NO: 10;
comprising a VH comprising a sequence as set forth in SEQ ID NO: 5 and a VL comprising a sequence as set forth in SEQ ID NO: 7;
comprising a VH comprising a sequence as set forth in SEQ ID NO: 5 and a VL comprising a sequence as set forth in SEQ ID NO: 8;
comprising a VH comprising a sequence as set forth in SEQ ID NO: 5 and a VL comprising a sequence as set forth in SEQ ID NO: 9; or
comprising a VH comprising a sequence as set forth in SEQ ID NO: 5 and a VL comprising a sequence as set forth in SEQ ID NO: 10.

6. The antigen binding protein of any one of claims 1 to 5, wherein the antigen binding domain comprises:
A. a VH comprising a complementarity determining region (CDR) 1 comprising or consisting of an amino acid sequence of SEQ ID NO: 12; a CDR2 comprising or consisting of an amino acid sequence of SEQ ID NO: 13; a CDR3 comprising or consisting of an amino acid sequence of SEQ ID NO: 14, and a VL comprising a complementarity determining region (CDR) 1 comprising or consisting of an amino acid sequence SEQ ID NO: 21; a CDR2 comprising or consisting of an amino acid sequence of SEQ ID NO: 22; a CDR3 comprising or consisting of an amino acid sequence SEQ ID NO: 23,
and any one of B., C., D., E., F., F., G., H., I., J., K., L., M., N., O., P., Q., R.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 65, 66, 67 and 68 respectively, and for VL FRs the reference sequences are set forth in SEQ ID NOs: 69, 70, 71 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 69, 70, 71 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 84, 85, 86 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 24, 26, 27 and 28 respectively,
or
F. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 16, 18 and 20 respectively, and for VL FRs the reference sequences are set forth in SEQ ID NOs: 24, 25, 27 and 28 respectively,
or
G. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 69, 70, 71 and 28 respectively,
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 84, 85, 86 and 28 respectively,
or
I. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 24, 26, 27 and 28 respectively,
or
J. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 17, 19 and 20 respectively, and for VL FRs the reference sequences are set forth in SEQ ID NOs: 24, 25, 27 and 28 respectively,
or
K. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 69, 70, 71 and 28 respectively,
or
L. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 84, 85, 86 and 28 respectively,
or
M. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 24, 26, 27 and 28 respectively,
or
N. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 16, 82 and 20 respectively, and for VL FRs the reference sequences are set forth in SEQ ID NOs: 24, 25, 27 and 28 respectively,
or
O. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 81, 83 and 20 respectively, and for VL FRs the reference sequences are set forth in SEQ ID NOs: 69, 70, 71 and 28 respectively,
or
P. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 81, 83 and 20 respectively, and for VL FRs the reference sequences are set forth in SEQ ID NOs: 84, 85, 86 and 28 respectively,
or
Q. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 81, 83 and 20 respectively, and for VL FRs the reference sequences are set forth in SEQ ID NOs: 24, 26, 27 and 28 respectively,
or
R. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 80, 81, 83 and 20 respectively, and for VL FRs the reference sequences are set forth in SEQ ID NOs: 24, 25, 27 and 28 respectively.

7. The antigen binding protein of any one of claims 1 to 5, wherein the antigen binding domain comprises:
A. a VH comprising a complementarity determining region CDR1 comprising or consisting of an amino acid sequence of SEQ ID NOs: 42 or 43; a CDR2 comprising or consisting of an amino acid sequence of SEQ ID NO: 44; a CDR3 comprising or consisting of an amino acid sequence of SEQ ID NO: 45, and a VL comprising complementarity determining region CDR1 comprising or consisting of an amino acid sequence of SEQ ID NO: 51; a CDR2 comprising or consisting of an amino acid sequence of SEQ ID NO: 52; a CDR3 comprising or consisting of an amino acid sequence of SEQ ID NO: 23,
and any one of B., C., D. or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 53, 55, 56 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 53, 54, 56 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 53, 55, 56 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 53, 54, 56 and 28 respectively.

8. The antigen binding protein of any one of claims 1 to 5, wherein the antigen binding domain comprises:
A. a VH comprising a complementarity determining region CDR1 comprising or consisting of an amino acid sequence of SEQ ID NOs: 42 or 43; a CDR2 comprising or consisting of an amino acid sequence of SEQ ID NO: 44; a CDR3 comprising or consisting of an amino acid sequence of SEQ ID NO: 45, and a VL comprising a complementarity determining region CDR1 comprising or consisting of an amino acid sequence of SEQ ID NO: 64; a CDR2 comprising or consisting of an amino acid sequence SEQ ID NO: 52; a CDR3 comprising or consisting of an amino acid sequence of SEQ ID NO: 23,
and any one of B., C., D., or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 75, 76, 77 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 47, 49 and 20 respectively, and for VL FRs the reference sequences are set forth in SEQ ID NOs: 92, 93, 94 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 75, 76, 77 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 46, 48, 50 and 20 respectively, and for VL FRs the reference sequences are set forth in SEQ ID NOs: 92, 93, 94 and 28 respectively.

9. The antigen binding protein of any one of claims 1 to 5, wherein the antigen binding domain comprises:
A. a VH comprising a complementarity determining region CDR1 comprising or consisting of an amino acid sequence of SEQ ID NOs: 42 or 87; a CDR2 comprising or consisting of an amino acid sequence of SEQ ID NO: 88; a CDR3 comprising or consisting of an amino acid sequence of SEQ ID NO: 45, and a VL comprising a complementarity determining region CDR1 comprising or consisting of an amino acid sequence of SEQ ID NO: 51; a CDR2 comprising or consisting of an amino acid sequence of SEQ ID NO: 52; a CDR3 comprising or consisting of an amino acid sequence of SEQ ID NO: 23,
and any one of B., C., D., or E:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 53, 55, 56 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 53, 54, 56 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 53, 55, 56 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 53, 54, 56 and 28 respectively.

10. The antigen binding protein of any one of claims 1 to 5, wherein the antigen binding domain comprises:
A. a VH comprising a complementarity determining region CDR1 comprising or consisting of an amino acid sequence of SEQ ID NOs: 42 or 87; a CDR2 comprising or consisting of an amino acid sequence of SEQ ID NO: 88; a CDR3 comprising or consisting of an amino acid sequence of SEQ ID NO: 45, and a VL comprising a complementarity determining region CDR1 comprising or consisting of an amino acid sequence of SEQ ID NO: 64; a CDR2 comprising or consisting of an amino acid sequence of SEQ ID NO: 52; a CDR3 comprising or consisting of an amino acid sequence of SEQ ID NO: 23,
and any one of B., C., D., or E.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 75, 76, 77 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 47, 90 and 20 respectively, and for VL FRs the reference sequences are set forth in SEQ ID NOs: 92, 93, 94 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 75, 76, 77 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 89, 48, 91 and 20 respectively, and for VL FRs the reference sequences are set forth in SEQ ID NOs: 92, 93, 94 and 28 respectively.

11. The antigen binding protein of any one of claims 1 to 5, wherein the antigen binding domain comprises:
A. a VH comprising a complementarity determining region CDR1 comprising or consisting of an amino acid sequence of SEQ ID NO: 57; a CDR2 comprising or consisting of an amino acid sequence of SEQ ID NO: 58; a CDR3 comprising or consisting of an amino acid sequence of SEQ ID NO: 45, and a VL comprising a complementarity determining region CDR1 comprising or consisting of an amino acid sequence of SEQ ID NO: 51; a CDR2 comprising or consisting of an amino acid sequence of SEQ ID NO: 52; a CDR3 comprising or consisting of an amino acid sequence of SEQ ID NO: 23,
and any one of B., C., D., E., F., G., H., or I.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 53, 55, 56 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 53, 54, 56 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 53, 55, 56 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 53, 54, 56 and 28 respectively,
or
F. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 53, 55, 56 and 28 respectively,
or
G. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 53, 54, 56 and 28 respectively,
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 53, 55, 56 and 28 respectively,
or
I. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 53, 54, 56 and 28 respectively.

12. The antigen binding protein of any one of claims 1 to 5, wherein the antigen binding domain comprises:
A. a VH comprising a complementarity determining region CDR1 comprising or consisting of an amino acid sequence of SEQ ID NO: 57; a CDR2 comprising or consisting of an amino acid sequence of SEQ ID NO: 58; a CDR3 comprising or consisting of an amino acid sequence of SEQ ID NO: 45, and a VL comprising a complementarity determining region CDR1 comprising or consisting of an amino acid sequence of SEQ ID NO: 64; a CDR2 comprising or consisting of an amino acid sequence of SEQ ID NO: 52; a CDR3 comprising or consisting of an amino acid sequence of SEQ ID NO: 23,
and any one of B., C., D., E., F., G., H., or I.:
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 75, 76, 77 and 28 respectively,
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 59, 61 and 20 respectively, and for VL FRs the reference sequences are set forth in SEQ ID NOs: 99, 100, 101 and 28 respectively,
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 75, 76, 77 and 28 respectively,
or
E. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 60, 62 and 20 respectively, and for VL FRs the reference sequences are set forth in SEQ ID NOs: 99, 100, 101 and 28 respectively,
or
F. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 75, 76, 77 and 28 respectively,
or
G. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 59, 97 and 20 respectively, and for VL FRs the reference sequences are set forth in SEQ ID NOs: 99, 100, 101 and 28 respectively,
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 75, 76, 77 and 28 respectively,
or
I. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 95, 96, 98 and 20 respectively, and for VL FRs the reference sequences are set forth in SEQ ID NOs: 99, 100, 101 and 28 respectively.

12. The antigen binding protein of any one of claims 1 to 5, wherein the antigen binding domain comprises:
A. a VH comprising a complementarity determining region (CDR) 1, CDR2 and CDR3 as defined in any of (o) above and a VL comprising a CDR1, CDR2 and CDR3 as defined in any of (p) above, and a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 72, 73, 74 and 68 respectively, and for VL FRs the reference sequences are set forth in SEQ ID NOs: 75, 76, 77 and 28 respectively.
or
B. a VH comprising a complementarity determining region (CDR) 1, CDR2 and CDR3 as defined in any of (q) above and a VL comprising a CDR1, CDR2 and CDR3 as defined in any of (r) above and a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NOs: 15, 78, 79 and 68 respectively, and for VL FRs the reference sequences are set forth in SEQ ID NOs: 75, 76, 77 and 28 respectively.

13. The antigen binding domain of any one of claims 1 to 12, wherein the antigen binding domain comprises a variable heavy chain comprising the amino acid sequence as set forth in any one of SEQ ID NOs: 2 to 5 and a variable light chain comprising the amino acid sequence as set forth in any one of SEQ ID NOs: 7 to 10.

14. The antigen binding domain of any one of claims 1 to 13, wherein the antigen binding protein is in the form of:
(i) a single domain antibody (sdAb);
(ii) a single chain Fv fragment (scFv);
(iii) a dimeric scFv (di-scFv);
(iv) one of (ii) or (iii) linked to a constant region of an antibody, Fc or a heavy chain constant domain (CH) 2 and/or CH3;
(v) one of (i) to (iv) linked to a protein that binds to an immune effector cell;
(vi) one of (i) to (iv) linked to a modified immune cell receptor, such as a modified T cell receptor; or
(vii) one of (i) to (iv) in the context of a chimeric antigen receptor (CAR) or variant T cell receptor.

15. The antigen binding domain of any one of claims 1 to 14, wherein the antigen binding protein is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3; or
(ix) one of (i) to (vii) linked to a protein that binds to an immune effector cell.
(x) one of (i) to (vii) linked to a protein that binds to an immune effector cell;
(xi) one of (i) to (vii) linked to a modified immune cell receptor, such as a modified T cell receptor; or
(xiii) one of (i) to (vii) in the context of a chimeric antigen receptor (CAR) or variant T cell receptor (including in the context of a universal CAR system for use with polypeptides comprising antigen binding domains for binding antigens on the surface of a target cell).

16. The antigen binding domain of any one of claims 1 to 15, wherein the antigen binding protein comprises, consists essentially of or consists of an amino acids sequence of (in order of N to C terminus or C to N terminus):
SEQ ID NO: 2 and 10;
SEQ ID NO: 3 and 9;
SEQ ID NO: 2 and 9;
SEQ ID NO: 2 and 7;
SEQ ID NO: 2 and 8;
SEQ ID NO: 3 and 7;
SEQ ID NO: 3 and 8;
SEQ ID NO: 3 and 10;
SEQ ID NO: 4 and 7;
SEQ ID NO: 4 and 8;
SEQ ID NO: 4 and 9;
SEQ ID NO: 4 and 10;
SEQ ID NO: 5 and 7;
SEQ ID NO: 5 and 8;
SEQ ID NO: 5 and 9; or
SEQ ID NO: 5 and 10.

17. A fusion protein comprising an antigen binding protein of any one of claims 1 to 16.

18. A chimeric antigen receptor or variant T cell receptor comprising an antigen binding protein of any one of claims 1 to 16, or a fusion protein of claim 17.

19. A conjugate comprising an antigen binding protein of any one of claims 1 to 16.

20. A nucleic acid encoding an antigen binding protein of any one of claims 1 to 16 or a fusion protein of claim 17.

21. A vector construct comprising a nucleic acid of claim 20.

22. A cell comprising a nucleic acid of claim 20 or a vector construct of claim 21.

23. A pharmaceutical composition comprising an antigen binding protein of any one of claims 1 to 16, a fusion protein of claim 17, or a conjugate of claim 19 and a pharmaceutically acceptable carrier, diluent or excipient.

24. A diagnostic composition comprising an antigen binding protein of any one of claims 1 to 16, a fusion protein of claim 17, or a conjugate of claim 19 and a pharmaceutically acceptable carrier, diluent or excipient.


SEQUENCE LISTING (101 sequences)

SEQ ID NO: 1 - protein, 117 aa, synthetic construct
QVQLQQSAGELARPGASVKMSCKASGYSFTTYTIHWVKQRPGQGLEWIGYINPSSGYAAYNQNFKDETTLTADKSSSTAYMQLSSLTSEDSAVYYCAREKFYGYDYWGQGTTLTVSS

SEQ ID NO: 2 - protein, 117 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 3 - protein, 117 aa, synthetic construct
QVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSS

SEQ ID NO: 4 - protein, 117 aa, synthetic construct
QVQLVQSGSELKKPGASVKVSCKASGYSFTTYTIHWVRQAPGQGLEWIGYINPSSGYAAYNQGFTGRFVLSADKSVSTAYLQISSLKAEDTAVYYCAREKFYGYDYWGQGTTVTVSS

SEQ ID NO: 5 - protein, 117 aa, synthetic construct
QVQLVQSGSELKKPGASVKVSCKASGYSFTTYTMNWVRQAPGQGLEWMGYINPSSGYAAYNQGFTGRFVFSADKSVSTAYLQISSLKAEDTAVYYCAREKFYGYDYWGQGTTVTVSS

SEQ ID NO: 6 - protein, 112 aa, synthetic construct
DVLMTQLPLSLPVSLGDQASISCRSSQSIVHRNGNTYLEWYLLKPGQSPKLLIYKVSNRFSGVPDRFSGSGSGTDFTLKISRVEAEDLGVYYCFQGSHVPYTFGGGTKLEIK

SEQ ID NO: 7 - protein, 112 aa, synthetic construct
DVVMTQSPLSLPVTLGQPASISCRSSQSIVHRNGNTYLEWFQLRPGQSPRLLIYKVSNRFSGVPDRFSGSGSGTDFTLKISRVEAEDVGVYYCFQGSHVPYTFGGGTKLEIK

SEQ ID NO: 8 - protein, 112 aa, synthetic construct
DVVMTQSPLSLPVTLGQPASISCRSSQSIVHRNGNTYLEWFQQRPGQSPRLLIYKVSNRFSGVPDRFSGSGSGTDFTLKISRVEAEDVGVYYCFQGSHVPYTFGGGTKLEIK

SEQ ID NO: 9 - protein, 112 aa, synthetic construct
DVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIK

SEQ ID NO: 10 - protein, 112 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 11 - protein, 4 aa, synthetic construct
DYKD

SEQ ID NO: 12 - protein, 8 aa, synthetic construct
GYSFTTYT

SEQ ID NO: 13 - protein, 8 aa, synthetic construct
INPSSGYA

SEQ ID NO: 14 - protein, 10 aa, synthetic construct
AREKFYGYDY

SEQ ID NO: 15 - protein, 25 aa, synthetic construct [sequence omitted]

SEQ ID NO: 16 - protein, 17 aa, synthetic construct
IHWVRQAPGQGLEWIGY

SEQ ID NO: 17 - protein, 17 aa, synthetic construct
MHWVRQAPGQGLEWMGY

SEQ ID NO: 18 - protein, 38 aa, synthetic construct
AYNQKFQGRVTLTADKSTSTAYMELSSLRSEDTAVYYC

SEQ ID NO: 19 - protein, 38 aa, synthetic construct
AYNQKFQGRVTMTADKSTSTAYMELSSLRSEDTAVYYC

SEQ ID NO: 20 - protein, 11 aa, synthetic construct
WGQGTTVTVSS

SEQ ID NO: 21 - protein, 11 aa, synthetic construct
QSIVHRNGNTY

SEQ ID NO: 22 - skipped

SEQ ID NO: 23 - protein, 9 aa, synthetic construct
FQGSHVPYT

SEQ ID NO: 24 - protein, 26 aa, synthetic construct
DVVMTQSPDSLAVSLGERATINCKSS

SEQ ID NO: 25 - protein, 17 aa, synthetic construct
LEWYQQKPGQPPKLLIY

SEQ ID NO: 26 - protein, 17 aa, synthetic construct
LEWYQLKPGQPPKLLIY

SEQ ID NO: 27 - protein, 36 aa, synthetic construct [sequence omitted]

SEQ ID NO: 28 - protein, 10 aa, synthetic construct
FGGGTKLEIK

SEQ ID NO: 29 - protein, 10 aa, synthetic construct
GDYKDDDDKG

SEQ ID NO: 30 - protein, 8 aa, synthetic construct
DYKDDDDK

SEQ ID NO: 31 - protein, 9 aa, synthetic construct
MDYKDDDDK

SEQ ID NO: 32 - protein, 7 aa, synthetic construct
DFKDDDK

SEQ ID NO: 33 - protein, 8 aa, synthetic construct
DYKAFDNL

SEQ ID NO: 34 - protein, 7 aa, synthetic construct
DYKDHDG

SEQ ID NO: 35 - protein, 9 aa, synthetic construct
MDFKDDDDK

SEQ ID NO: 36 - protein, 9 aa, synthetic construct
MDYKAFDNL

SEQ ID NO: 37 - protein, 7 aa, synthetic construct
DYKDHDI

SEQ ID NO: 38 - protein, 5 aa, synthetic construct
DYKDH

SEQ ID NO: 39 - protein, 5 aa, synthetic construct
DYKDD

SEQ ID NO: 40 - protein, 6 aa, synthetic construct
DYKDHD

SEQ ID NO: 41 - protein, 6 aa, synthetic construct
DYKDDD

SEQ ID NO: 42 - protein, 5 aa, synthetic construct
TYTIH

SEQ ID NO: 43 - protein, 5 aa, synthetic construct
TYTMH

SEQ ID NO: 44 - protein, 17 aa, synthetic construct
YINPSSGYAAYNQKFQG

SEQ ID NO: 45 - protein, 8 aa, synthetic construct
EKFYGYDY

SEQ ID NO: 46 - protein, 29 aa, synthetic construct
QVQLVQSGAEVKKPGASVKVSCKASGYSF

SEQ ID NO: 47 - protein, 14 aa, synthetic construct
WVRQAPGQGLEWIG

SEQ ID NO: 48 - protein, 14 aa, synthetic construct
WVRQAPGQGLEWMG

SEQ ID NO: 49 - protein, 32 aa, synthetic construct
RVTLTADKSTSTAYMELSSLRSEDTAVYYCAR

SEQ ID NO: 50 - protein, 32 aa, synthetic construct
RVTMTADKSTSTAYMELSSLRSEDTAVYYCAR

SEQ ID NO: 51 - protein, 16 aa, synthetic construct
KSSQSIVHRNGNTYLE

SEQ ID NO: 52 - protein, 7 aa, synthetic construct
KVSNRFS

SEQ ID NO: 53 - protein, 23 aa, synthetic construct
DVVMTQSPDSLAVSLGERATINC

SEQ ID NO: 54 - protein, 15 aa, synthetic construct
WYQQKPGQPPKLLIY

SEQ ID NO: 55 - protein, 15 aa, synthetic construct
WYQLKPGQPPKLLIY

SEQ ID NO: 56 - protein, 32 aa, synthetic construct
GVPDRFSGSGSGTDFTLTISSLQAEDVAVYYC

SEQ ID NO: 57 - protein, 7 aa, synthetic construct
GYSFTTY

SEQ ID NO: 58 - protein, 6 aa, synthetic construct
NPSSGY

SEQ ID NO: 59 - protein, 19 aa, synthetic construct
TIHWVRQAPGQGLEWIGYI

SEQ ID NO: 60 - protein, 19 aa, synthetic construct
TMHWVRQAPGQGLEWMGYI

SEQ ID NO: 61 - protein, 41 aa, synthetic construct
AAYNQKFQGRVTLTADKSTSTAYMELSSLRSEDTAVYYCAR

SEQ ID NO: 62 - protein, 41 aa, synthetic construct
AAYNQKFQGRVTMTADKSTSTAYMELSSLRSEDTAVYYCAR

SEQ ID NO: 63 - protein, 17 aa, synthetic construct
YINPSSGYAAYNQNFKD

SEQ ID NO: 64 - protein, 16 aa, synthetic construct
RSSQSIVHRNGNTYLE

SEQ ID NO: 65 - protein, 25 aa, synthetic construct
QVQLQQSAGELARPGASVKMSCKAS

SEQ ID NO: 66 - protein, 17 aa, synthetic construct
IHWVKQRPGQGLEWIGY

SEQ ID NO: 67 - protein, 38 aa, synthetic construct
AYNQNFKDETTLTADKSSSTAYMQLSSLTSEDSAVYYC

SEQ ID NO: 68 - protein, 11 aa, synthetic construct
WGQGTTLTVSS

SEQ ID NO: 69 - protein, 26 aa, synthetic construct
DVLMTQLPLSLPVSLGDQASISCRSS

SEQ ID NO: 70 - protein, 17 aa, synthetic construct
LEWYLLKPGQSPKLLIY

SEQ ID NO: 71 - protein, 36 aa, synthetic construct
NRFSGVPDRFSGSGSGTDFTLKISRVEAEDLGVYYC

SEQ ID NO: 72 - protein, 30 aa, synthetic construct
QVQLQQSAGELARPGASVKMSCKASGYSFT

SEQ ID NO: 73 - protein, 14 aa, synthetic construct
WVKQRPGQGLEWIG

SEQ ID NO: 74 - protein, 32 aa, synthetic construct
ETTLTADKSSSTAYMQLSSLTSEDSAVYYCAR

SEQ ID NO: 75 - protein, 23 aa, synthetic construct
DVLMTQLPLSLPVSLGDQASISC

SEQ ID NO: 76 - protein, 15 aa, synthetic construct
WYLLKPGQSPKLLIY

SEQ ID NO: 77 - protein, 33 aa, synthetic construct
GVPDRFSGSGSGTDFTLKISRVEAEDLGVYYCF

SEQ ID NO: 78 - protein, 19 aa, synthetic construct
TIHWVKQRPGQGLEWIGYI

SEQ ID NO: 79 - protein, 41 aa, synthetic construct
AAYNQNFKDETTLTADKSSSTAYMQLSSLTSEDSAVYYCAR

SEQ ID NO: 80 - protein, 25 aa, synthetic construct
QVQLVQSGSELKKPGASVKVSCKAS

SEQ ID NO: 81 - protein, 17 aa, synthetic construct
MNWVRQAPGQGLEWMGY

SEQ ID NO: 82 - protein, 38 aa, synthetic construct
AYNQGFTGRFVLSADKSVSTAYLQISSLKAEDTAVYYC

SEQ ID NO: 83 - protein, 38 aa, synthetic construct
AYNQGFTGRFVFSADKSVSTAYLQISSLKAEDTAVYYC

SEQ ID NO: 84 - protein, 26 aa, synthetic construct
DVVMTQSPLSLPVTLGQPASISCRSS

SEQ ID NO: 85 - protein, 17 aa, synthetic construct
LEWFQQRPGQSPRLLIY

SEQ ID NO: 86 - protein, 36 aa, synthetic construct
NRFSGVPDRFSGSGSGTDFTLKISRVEAEDVGVYYC

SEQ ID NO: 87 - protein, 5 aa, synthetic construct
TYTMN

SEQ ID NO: 88 - protein, 17 aa, synthetic construct
YINPSSGYAAYNQGFTG

SEQ ID NO: 89 - protein, 30 aa, synthetic construct
QVQLVQSGSELKKPGASVKVSCKASGYSFT

SEQ ID NO: 90 - protein, 32 aa, synthetic construct
RFVLSADKSVSTAYLQISSLKAEDTAVYYCAR

SEQ ID NO: 91 - protein, 32 aa, synthetic construct
RFVFSADKSVSTAYLQISSLKAEDTAVYYCAR

SEQ ID NO: 92 - protein, 23 aa, synthetic construct
DVVMTQSPLSLPVTLGQPASISC

SEQ ID NO: 93 - protein, 15 aa, synthetic construct
WFQQRPGQSPRLLIY

SEQ ID NO: 94 - protein, 32 aa, synthetic construct
GVPDRFSGSGSGTDFTLKISRVEAEDVGVYYC

SEQ ID NO: 95 - protein, 25 aa, synthetic construct
QVQLVQSGSELKKPGASVKVSCKAS

SEQ ID NO: 96 - protein, 19 aa, synthetic construct
TMNWVRQAPGQGLEWMGYI

SEQ ID NO: 97 - protein, 41 aa, synthetic construct
AAYNQGFTGRFVLSADKSVSTAYLQISSLKAEDTAVYYCAR

SEQ ID NO: 98 - protein, 41 aa, synthetic construct
AAYNQGFTGRFVFSADKSVSTAYLQISSLKAEDTAVYYCAR

SEQ ID NO: 99 - protein, 23 aa, synthetic construct
DVVMTQSPLSLPVTLGQPASISC

SEQ ID NO: 100 - protein, 15 aa, synthetic construct
WFQQRPGQSPRLLIY

SEQ ID NO: 101 - protein, 32 aa, synthetic construct; identical to SEQ ID NO: 94